(19) United States
(12) Patent Application Publication
(10) Pub. No.: US 2015/0367130 A1
(43) Pub. Date: Dec. 24, 2015

(54) INTERNAL PRESSURE MANAGEMENT SYSTEM

(71) Applicant: Cochlear Limited, Macquarie University (AU)

(72) Inventors: Joris WALRAEVENS, Mechelen (BE); Pieter WISKERKE, Antwerpen (BE); Francesca PARIS, Mechelen (BE); Alexander HUBER, Zurich (CH); Lukas PROCHAZKA, Zurich (CH); Andrin LANDOLT, Zurich (CH); Dominik OBRIST, Zurich (CH)

(21) Appl. No.: 14/548,714

(22) Filed: Nov. 20, 2014

Related U.S. Application Data
(60) Provisional application No. 62/013,829, filed on Jun. 18, 2014.

(51) Int. Cl. A61N 1/36; H04R 25/00

(52) U.S. Cl. CPC A61N 1/36032; H04R 25/65; H04R 2225/67; H04R 2225/61

(57) ABSTRACT

A device including an implantable sensor having a membrane displaceable in response to physical phenomena outside the sensor, wherein the device is configured to equalize a static pressure difference between an ambient environment and a back volume of the sensor.

This application claims priority to Provisional U.S. Patent Application No. 62/013,829, entitled INTERNAL PRESSURE MANAGEMENT SYSTEM, filed on Jun. 18, 2014, naming Joris WALRAEVENS of Mechelen, Belgium, as an inventor, the entire contents of that application being incorporated herein by reference in its entirety.

### BACKGROUND

Hearing loss, which may be due to many different causes, is generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound. One example of a hearing prosthesis is a cochlear implant.

Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from conductive hearing loss typically receive an acoustic hearing aid. Hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the auditory nerve.

In contrast to hearing aids, which rely primarily on the principles of air conduction, certain types of hearing prostheses commonly referred to as cochlear implants convert a received sound into electrical stimulation. The electrical stimulation is applied to the cochlea, which results in the perception of the received sound.

### SUMMARY

In an exemplary embodiment, there is a device, comprising an implantable sensor having a membrane displaceable in response to physical phenomena outside the sensor, wherein the device is configured to equalize a static pressure difference between an ambient environment and a back volume of the sensor.

In another exemplary embodiment, there is a device, comprising an implantable microphone having a membrane displaceable in response to a change in a phenomena of fluid in a cochlea induced by ambient sound, the membrane forming a portion of a boundary of a back volume of the microphone, wherein the device is configured to expand and contract a size of the volume of the back volume independent of movement of the membrane.

In another exemplary embodiment, there is a device comprising an implantable static pressure equalization system configured to equalize an internal pressure of an apparatus with a static pressure of an ambient environment, the apparatus being configured to sense a dynamic phenomenon in a recipient, the system including at least one diaphragm bounding a volume, wherein the diaphragm is configured to deflect in response to a change in the static pressure, thereby adjusting the size of the volume bounded by the diaphragm, wherein the system is configured such that the volume is placed in fluid communication with the apparatus, and wherein the diaphragm is sheltered by at least two substantially rigid components located on opposite sides of the diaphragm in a direction normal to a maximum diameter of the diaphragm.

In another exemplary embodiment, there is a method, comprising, automatically maintaining a neutral position of at least one of (i) a membrane of an implanted microphone having a front volume and a back volume separated by the membrane and fluidically isolated from one another in response to a change in pressure of the front volume induced by a change in pressure of an ambient environment in which the microphone is located or (ii) a flexible diaphragm of a pressure receptor that hermetically isolates an internal volume in fluid communication with the microphone with an ambient environment by automatically adjusting the size of the back volume to at least substantially equalize the pressure of at least one of the back volume and the pressure of a combined front and back volume with the pressure of the ambient environment. In an exemplary embodiment, the method is executed in a cochlear implant implanted in a recipient, wherein the changes in the ambient environment correspond to changes in a pressure of fluid inside the cochlea of the recipient. In an exemplary embodiment, at least a portion of the back volume is located remote from the front volume.

### BRIEF DESCRIPTION OF THE DRAWINGS

Embodiments of the present invention are described below with reference to the attached drawings, in which:

FIG. 1A is a perspective view of an exemplary hearing prosthesis utilized in some exemplary embodiments;

FIG. 1B is a side view of the implantable components of the cochlear implant illustrated in FIG. 1A;

FIG. 2 is a side view of an embodiment of the electrode array illustrated in FIGS. 1A and 1B in a curled orientation;

FIG. 3A is a side view of an exemplary electrode array assembly according to an embodiment;

FIG. 3B is a conceptual side view of the exemplary electrode array of FIG. 3A inserted into a cochlea;

FIG. 4 is an isometric view of a sensor according to an exemplary embodiment;

FIG. 5 is a functional schematic of an exemplary embodiment;

FIG. 6 is another functional schematic of an alternate exemplary embodiment;

FIG. 7A is a schematic of a portion of a sensor according to an exemplary embodiment;

FIG. 7B is a schematic of an adaptive volume structure that is connected to the portion of the sensor depicted in FIG. 7A;

FIG. 7C is a schematic depicting additional details of the adaptive volume structure of FIG. 7B;

FIG. 8 is a schematic of an alternative embodiment of an adaptive volume structure according to an exemplary embodiment;

FIG. 9 is a schematic of another alternative embodiment of an adaptive volume structure according to an exemplary embodiment;

FIG. 10 is a schematic of another alternative embodiment of an adaptive volume structure according to an exemplary embodiment;

FIG. 11 is a schematic of a cochlear implant implementing the embodiment of FIGS. 9 and 10;

FIG. 12 is a schematic of a portion of a sensor according to an exemplary embodiment including an integral adaptive volume structure;

FIG. 13 is a schematic of a portion of a sensor according to an exemplary embodiment including an integral adaptive volume structure;

FIG. 14 is a schematic of a cross-sectional view of a portion of a micro tube according to an exemplary embodiment;

FIG. 15A is an isometric view of an exemplary micro tube according to FIG. 14; and

FIG. 15B is a schematic of a portion of the portion of the micro tube of FIG. 14 depicting a functional aspect associated with flexing thereof; and

FIGS. 16-20 present graphs of performance data for some exemplary embodiments.

### DETAILED DESCRIPTION

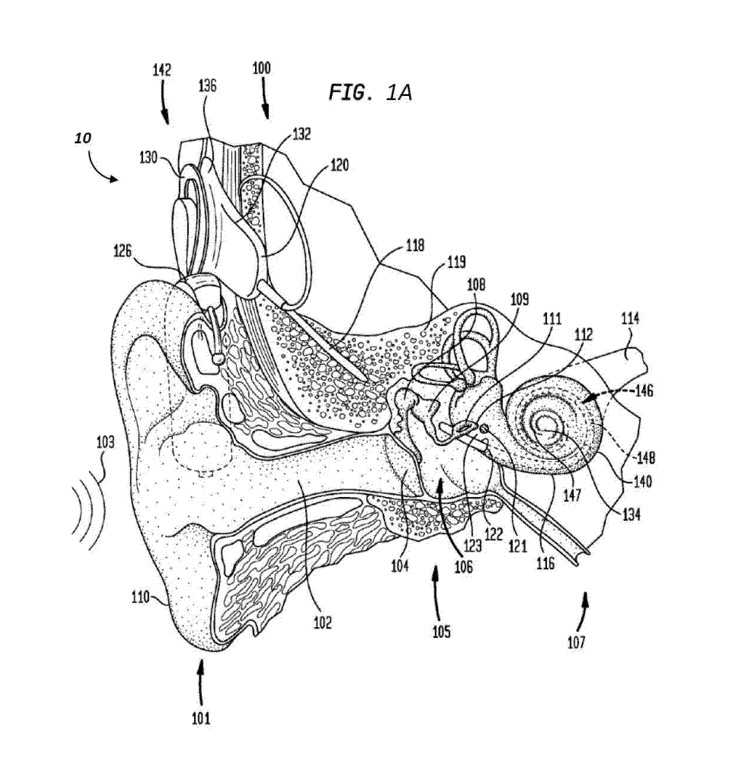

FIG. 1A is perspective view of a totally implantable cochlear implant, referred to as cochlear implant 100, implanted in a recipient. The totally implantable cochlear implant 100 is part of a system 10 that can include external components, as will be detailed below.

The recipient has an outer ear 101, a middle ear 105 and an inner ear 107. Components of outer ear 101, middle ear 105 and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant.

In the illustrative arrangement of FIG. 1A, external device 142 may comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments of the present invention.

Cochlear implant 100 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate stimulating assembly 118. In embodiments of the present invention, internal energy transfer assembly 132 and main implantable component 120 are hermetically sealed within a biocompatible housing. In embodiments of the present invention, main implantable component 120 includes a sound processing unit (not shown) to convert the sound signals received by the implantable microphone in internal energy transfer assembly 132 to data signals. Main implantable component 120 further includes a stimulator unit (also not shown) which generates electrical stimulation signals based on the data signals. The electrical stimulation signals are delivered to the recipient via elongate stimulating assembly 118.

Elongate stimulating assembly 118 has a proximal end connected to main implantable component 120, and a distal end implanted in cochlea 140. Stimulating assembly 118 extends from main implantable component 120 to cochlea 140 through mastoid bone 119. In some embodiments stimulating assembly 118 may be implanted at least in basal region 116, and sometimes further. For example, stimulating assembly 118 may extend towards apical end of cochlea 140, referred to as cochlea apex 134. In certain circumstances, stimulating assembly 118 may be inserted into cochlea 140 via a cochleostomy 122. In other circumstances, a cochleostomy may be formed through round window 121, oval window 112, the promontory 123 or through an apical turn 147 of cochlea 140.

Stimulating assembly 118 comprises a longitudinally aligned and distally extending array 146 of electrodes 148, disposed along a length thereof. As noted, a stimulator unit generates stimulation signals which are applied by stimulating contacts 148, which, in an exemplary embodiment, are electrodes, to cochlea 140, thereby stimulating auditory nerve 114. In an exemplary embodiment, stimulation contacts can be any type of component that stimulates the cochlea (e.g., mechanical components, such as piezoelectric devices that move or vibrate, thus stimulating the cochlea (e.g., by inducing movement of the fluid in the cochlea), electrodes that apply current to the cochlea, etc.). Embodiments detailed herein will generally be described in terms of an electrode assembly 118 utilizing electrodes as elements 148. It is noted that alternate embodiments can utilize other types of stimulating devices. Any device, system or method of stimulating the cochlea can be utilized in at least some embodiments.

As noted, cochlear implant 100 comprises a totally implantable prosthesis that is capable of operating, at least for a period of time, without the need for external device 142. Therefore, cochlear implant 100 further comprises a rechargeable power source (not shown) that stores power received from external device 142. The power source may comprise, for example, a rechargeable battery. During operation of cochlear implant 100, the power stored by the power source is distributed to the various other implanted components as needed. The power source may be located in main implantable component 120, or disposed in a separate implanted location.

It is noted that the teachings detailed herein and/or variations thereof can be utilized with a non-totally implantable prosthesis. That is, in an alternate embodiment of the cochlear implant 100, the cochlear implant 100 is a traditional hearing prosthesis.

While various aspects of the present invention are described with reference to a cochlear implant (whether it be a device utilizing electrodes or stimulating contacts that impart vibration and/or mechanical fluid movement within the cochle), it will be understood that various aspects of the embodiments detailed herein are equally applicable to other stimulating medical devices having an array of electrical simulating electrodes such as auditory brain implant (ABI), functional electrical stimulation (FES), spinal cord stimulation (SCS), penetrating ABI electrodes (PABI), and so on. Further, it should be appreciated that the present invention is applicable to stimulating medical devices having electrical stimulating electrodes of all types such as straight electrodes, peri-modiolar electrodes and short/basilar electrodes. Also, various aspects of the embodiments detailed herein and/or variations thereof are applicable to devices that are non-stimulating and/or have functionality different from stimulating tissue, such as for, example, any intra-body dynamic phenomenon (e.g., pressure, or other phenomenon consistent with the teachings detailed herein) measurement/sensing, etc., which can include use of these teachings to sense or otherwise detect a phenomenon at a location other than the cochlea (e.g., within a cavity containing the brain, the heart, etc.). Additional embodiments are applicable to bone conduction devices, Direct Acoustic Cochlear Stimulators/Middle Ear Prostheses, and conventional acoustic hearing aids. Any device, system or method of evoking a hearing percept can be used in conjunction with the teachings detailed herein.

FIG. 1B is a side view of the internal component of cochlear implant 100 without the other components of system 10 (e.g., the external components). Cochlear implant 100 comprises a receiver/stimulator 180 (combination of main implantable component 120 and internal energy transfer assembly 132) and an stimulating assembly or lead 118. Stimulating assembly 118 includes a helix region 182, a transition region 184, a proximal region 186, and an intra-cochlear region 188. Proximal region 186 and intra-cochlear region 188 form an electrode array assembly 190. In an exemplary embodiment, proximal region 186 is located in the middle-ear cavity of the recipient after implantation of the intra-cochlear region 188 into the cochlea. Thus, proximal region 186 corresponds to a middle-ear cavity sub-section of the electrode array assembly 190. Electrode array assembly 190, and in particular, intra-cochlear region 188 of electrode array assembly 190, supports a plurality of electrode contacts 148. These electrode contacts 148 are each connected to a respective conductive pathway, such as wires, PCB traces, etc. (not shown) which are connected through lead 118 to receiver/stimulator 180, through which respective stimulating electrical signals for each electrode contact 148 travel.

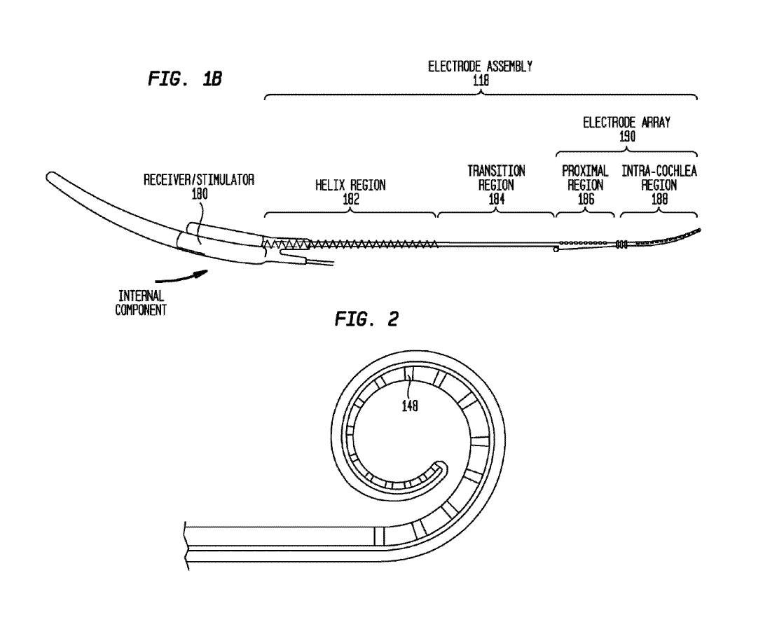

FIG. 2 is a side view of electrode array assembly 190 in a curled orientation, as it would be when inserted in a recipient's cochlea, with electrode contacts 148 located on the inside of the curve. FIG. 2 depicts the electrode array of FIG. 1B in situ in a patient's cochlea 140.

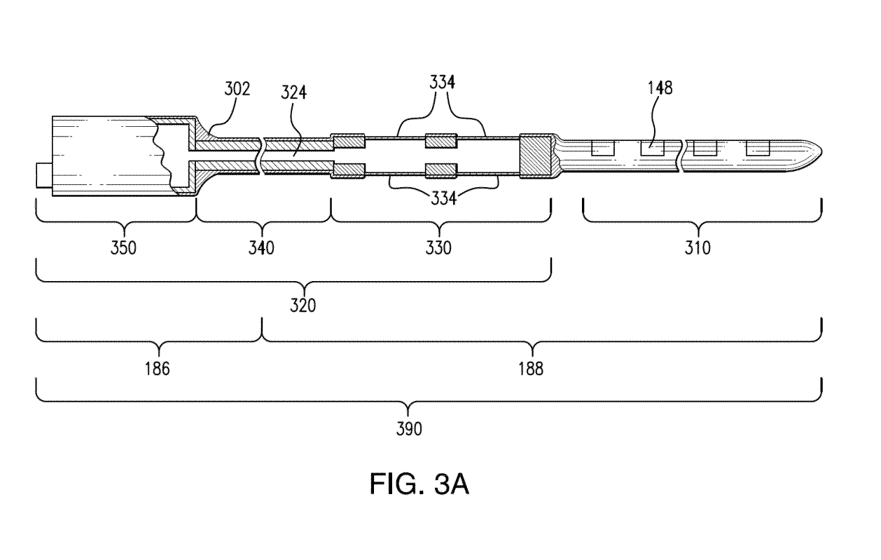

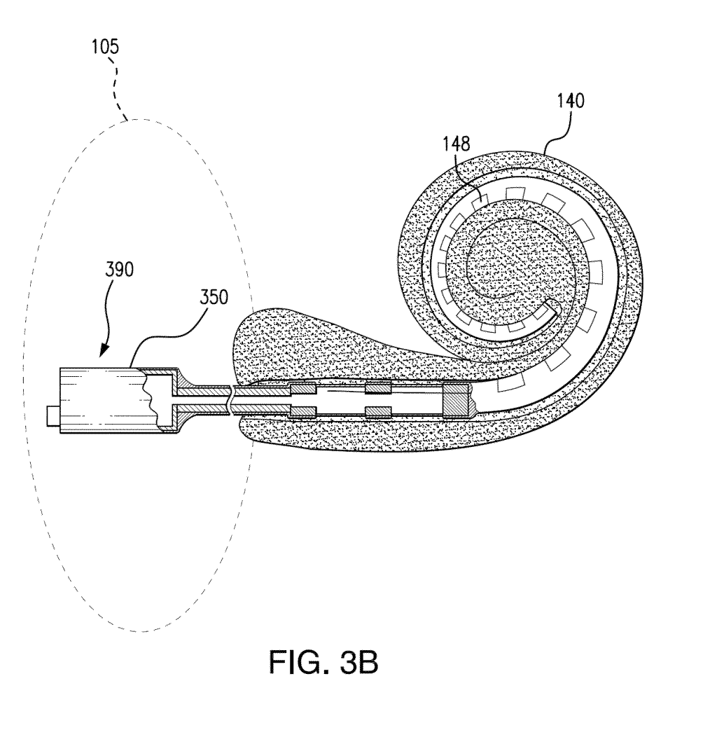

FIG. 3A depicts a side view of a device 390 corresponding to a cochlear implant electrode array assembly that can include some or all of the features of electrode array assembly 190 of FIG. 1B. More specifically, in an exemplary embodiment, stimulating assembly 118 includes electrode array assembly 390 instead of electrode array assembly 190 (i.e., 190 is replaced with 390).

Electrode array assembly 390 includes a cochlear implant electrode array 310 and an apparatus 320 configured to sense a phenomenon of the fluid in a cochlea. In an exemplary embodiment, electrode array assembly 390 has some and/or all of the functionality of electrode array assembly 190, where electrode array assembly 190 corresponds to a state-of-the-art electrode array assembly and/or variations thereof and/or an earlier model electrode array assembly. By way of example only and not by way of limitation, electrode array assembly 390 includes any electrode array 310 comprising a plurality of electrodes 148. The electrode array assembly 390 is configured such that the electrodes 148 of the electrode array 310 are in and/or can be placed in signal communication with the receiver stimulator 180.

In some embodiments, the phenomenon sensed by the apparatus 320 is a pressure of the fluid in the cochlea and/or a change in pressure of the fluid in the cochlea (a dynamic pressure). In an exemplary embodiment of FIG. 3A, the apparatus 320 is a pressure sensor assembly. Along these lines, in an exemplary embodiment, by way of example only and not by way of limitation, the apparatus 320 has the exemplary functionality of sensing pressure and/or pressure variations in fluid in the cochlea caused by vibrations impinging upon the outside of the cochlea and transmitted therein (e.g., through the oval window via ossicular vibrations (natural and/or prosthetically based), through the round window in scenarios where for whatever reason the round window transfers vibrations into the cochlea, and/or through any other part of the cochlea such that the cochlear fluid vibrates in a manner that the teachings detailed herein and/or variations thereof can be practiced). In at least some exemplary scenarios, the vibrations that impinge upon the outside of the cochlea and are transmitted therein are vibrations based on an ambient sound that would otherwise ultimately evoke a hearing percept in a normal hearing person. Accordingly, in an exemplary embodiment, the apparatus 320 is configured to utilize one or more phenomena of fluid in the cochlea associated with normal hearing and output a signal indicative of that phenomenon, where the outputted signal is based on ambient sound that caused or otherwise resulted in the one or more phenomena.

More particularly, apparatus 320 includes a physical phenomenon receptor 330 which is in fluid communication with conduit 340 which in turn is in fluid communication with sensor assembly 350. FIG. 3B depicts a conceptual representation of the electrode array assembly 390 inserted into a cochlea 140 that is configured to prosthetically remain in the cochlea (that is, it is configured to remain in the cochlea for a time period concomitant with the use of a prosthetic device, as opposed to a temporary insertion such as might be the case for a needle or the like). FIG. 3B depicts a conceptual drawing depicting the intra-cochlea region 188 of the electrode array assembly 390 in the cochlea 140, and the proximal region 186 of the electrode array assembly 390 located outside the cochlea 140, where the conduit 340 of the apparatus 320 extends from inside the cochlea 140 to outside the cochlea into the middle ear cavity, which is functionally represented by the dashed enclosure 105. It is noted that this drawing in FIG. 3B is just that conceptual, and is provided at least for the purpose of presenting the concept of the cochlear implant electrode array having apparatus 320 that is only partially inserted into the cochlea. In an exemplary embodiment, the electrode array assembly along with the receptor is inserted into the scala tympani. That said, in an alternate embodiment, at least the receptor is inserted into the scala vestibule. Accordingly, in an exemplary embodiment, there is an electrode array assembly configured such that the electrode array is insertable into the scala tympani, and the receptor is insertable into the scala vestibule. In an exemplary embodiment, the entire electrode array assembly is configured to be insertable into the scala vestibule. In yet another alternate embodiment, the receptor can be inserted into the tympani and the electrode array is insertable into the vestibule. Any method of utilizing the devices detailed herein and/or variations thereof that will enable the teachings detailed herein and/or variations thereof to be practiced can be utilized in at least some embodiments.

In an exemplary embodiment, the receptor 330 is a pressure receptor. In a non-mutually exclusive fashion, the receptor 330 can be a vibration receptor. As noted above, receptor 330 is a physical phenomenon receptor. Accordingly, in some embodiments, receptor 330 corresponds to any type of receptor that can function as a physical phenomenon receptor providing that the teachings detailed herein and/or variations thereof can be practiced with that receptor.

In the exemplary embodiment of the figures, the receptor 330 is a titanium cylinder having a closed end (distal end) and an end (proximal end) that is open via a port. The port provides fluid communication between the inside of the cylinder and the outside of the cylinder. Receptor 330 includes four diaphragms 334 arrayed about the longitudinal surface of the cylinder. In the embodiments of the figures, the diaphragms 334 cover through holes that extend through the longitudinal surface of the cylinder. The diaphragms 334 hermetically seal these holes. The diaphragms 334 configured to deflect or otherwise move as a result of pressure variations and/or vibrations impinging thereupon that are communicated thereto via the cochlea fluid. This causes pressure fluctuations within the receptor 330. In an exemplary embodiment, this is because the deflections of one or more diaphragms 334 change the volume within the receptor 330. Depending on the fluid that fills or otherwise is located in the receptor 330, vibrations can travel through the diaphragms from the cochlea fluid into the fluid inside the receptor 330.

Conduit 340 extends from receptor 330 to sensor assembly 350, and includes lumen 324 which places the inside of receptor 330 into fluid communication with the sensor assembly 350. In an exemplary embodiment, conduit 340 is a tube. Conduit 340 can be flexible and/or rigid. In an exemplary embodiment conduit 340 can be made of titanium. In an exemplary embodiment, in addition to the functionality of placing the receptor into fluid communication with the sensor assembly, conduit 340 has the functionality of maintaining a set/specific/control distance between the sensor assembly 350 (or more accurately, components of the sensor assembly 350 detail below) and the receptor 330. Still further, an exemplary embodiment, conduit 340 provides the transition between the intra-cochlea region 188 and the proximal region 186 of the electrode array assembly 390. In at least some embodiments, while not depicted in the figures, conduit 340 can include other components that have utilitarian value with respect to the tissue-electrode array interface (e.g. ribs, occluding features, antiviral and/or bacterial features etc.).

With respect to the embodiments detailed above, pressure variations and/or vibrations in the cochlea fluid that impinge upon the diaphragms deflect the diaphragms such that pressure fluctuations exist in/vibrations travel thorough the fluid-filled volume (e.g., a gas-filled volume, such as an inert gas such as argon-filled volume, etc.) that corresponds to the interior of the receptor 330 and the conduit 340, as well as the pertinent portions of the sensor assembly 350, in which resides a transducer that converts these pressure fluctuations/vibrations into another form of energy (e.g., electrical signal, an optical signal etc.), which in turn is ultimately provided (directly and/or indirectly) to the receiver stimulator 180 of the cochlear implant 100, which in turn interprets this energy as sound information Some details of the sensor assembly 350 will now be described.

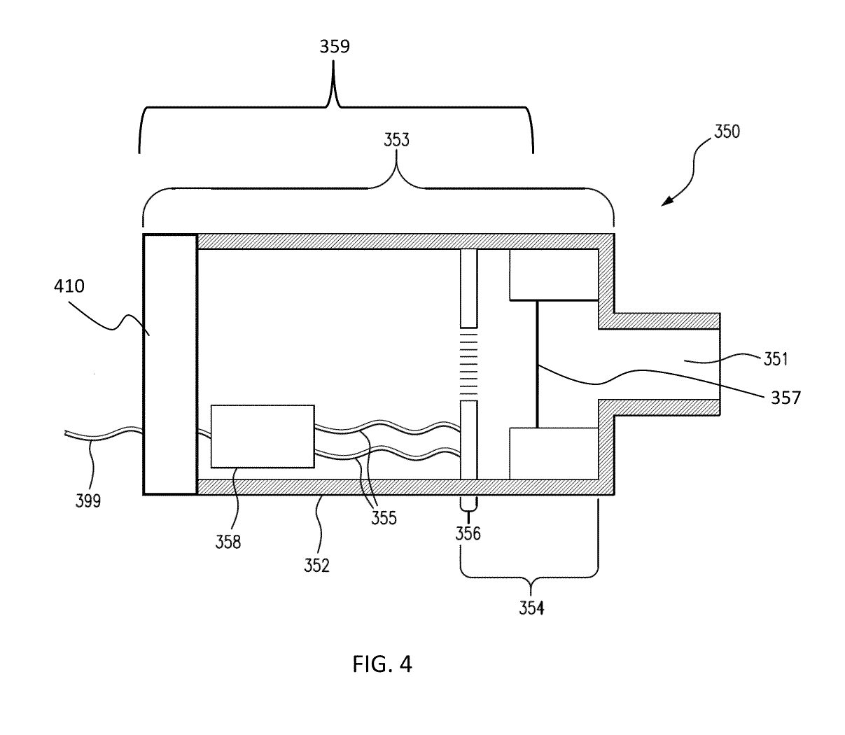

FIG. 4 depicts a cross-sectional view of an exemplary sensor assembly 350 in quasi-black-box format (some back lines are not shown for clarity). The sensor assembly 350 includes an enclosed bifurcated volume 353 established by housing 352 and black box 410 that is fluidly sealed (in some embodiments medically sealed and/or hermetically sealed) with the exception of port 351. As can be seen, port 351 is a male projection from the housing 352 having a hollow interior that is in fluid communication with the interior of the housing 352.

Housing 352 can be a hollow cylindrical body made of titanium or another biocompatible material. The housing 352 can be made of one or more such materials (e.g. it can be made of entirely titanium and/or a titanium alloy, or can be made out of different materials). The sensor assembly 350 includes a MEMS (micro-electro-mechanical system) condenser microphone 354 including a membrane 357 that bifurcates the volume 353 into a front volume (the volume to the right (relative to the orientation of FIG. 4) of membrane 357) and a back volume (the volume to the left (relative to the orientation of FIG. 4) of membrane 357. Reference numeral 359 indicates the back volume of the sensor assembly 350. Thus, the membrane 357 forms a portion of a boundary of a back volume of the microphone 354.

The sensor assembly 350 further includes a perforated backplate 356 which in at least some embodiments is part of the microphone 354 (it is noted that in some alternate embodiments, the back plate 356 is located in the front volume (i.e., to the right of the membrane 357)). In the embodiment of the figures, the microphone 354 is in fluid communication with the lumen 324 of conduit 340, which as noted above is in fluid communication with the interior of the receptor 330. Thus, in the embodiments of the figures, pressure changes inside the receptor 330 are fluidly communicated to the microphone 354.

In an exemplary embodiment, membrane 357 (also sometimes referred to as a diaphragm) is a pressure-sensitive membrane (diaphragm) that is etched directly onto a silicon chip. In this regard, the microphone falls within the rubric of “pressure sensor.” The pressure changes that occur inside receptor 330 as a result of the pressure changes in the cochlea fluid are sensed by the microphone 354. The microphone outputs the signals via electrical leads 355 to a pre-amplifier 358. The pre-amplifier 358, in at least some embodiments, amplifies the signal and/or lowers the noise of the microphone 354 and/or the output impedance of the microphone 354 that exists, in at least some embodiments, owing to the relatively large output impedance of the microphone 354. This lowering of the noise is relative to that which would be the case in the absence of the amplifier. It is noted that in some alternate embodiments, the preamplifier 358 is part of the MEMS microphone 354. In an exemplary embodiment, an A/D converter is integrated in the sensor assembly 350. In the embodiment depicted in FIG. 4, the preamplifier 358 is located inside the volume of the housing (in the back volume in particular). In an alternate embodiment, the preamplifier 358 is located outside the volume of the housing and/or outside the back volume and/or outside the front volume.

In an exemplary embodiment, the microphone is a MQM 31692 or a 32325 Knowles microphone or an ADMP504 microphone. (Any microphone that can enable the teachings detailed herein and/or variations thereof to be practiced can be utilized in at least some embodiments. In an exemplary embodiment, the microphone 354 (sensor) is a so-called air backed sensor. That said, in at least some exemplary embodiments, a so-called water backed sensor (or liquid backed sensor) can be utilized. Accordingly in an exemplary embodiment, the medium which fills the interior cavity of the apparatus 320 can be a liquid.

It is further noted that in alternate embodiments, the microphone 354 can be a MEMS microphone of a different species than the condenser microphone. In an exemplary embodiment, any MEMS-based membrane type sensor can be utilized such as by way of example, a capacitive, an optical, a piezoelectric membrane type sensor etc. Further, in an alternate embodiment, the microphone 354 need not be MEMS based. Any device, system, and/or method, that can transduce the pressure changes inside the closed system of the apparatus 320 can be utilized in at least some embodiments, providing that the teachings detailed herein and/or variations thereof can be practiced.

The microphone 354 transduces the pressure variations and outputs the transduced energy via electrical lead(s) 399. Via electrical lead(s) 399, the output of the microphone is received by the receiver stimulator 180 of the cochlear implant 100. In some embodiments, the sound processor of the cochlear implant 100 (the sound processor is typically located in the receiver stimulator 180 or in an implantable sound processor housing remote from the receiver stimulator 180 but in signal communication with the stimulator 180) receives the output of the microphone 354 or signal indicative of the output of the microphone 354, and processes that output into a signal (including a plurality of signals) that are used by the stimulator 180 to formulate output signal to the electrode array of the electrode array assembly to electrically stimulate the cochlea and evoke a hearing percept. In the exemplary embodiment as just described, the electrode array assembly 390 is utilized in a so-called totally implantable hearing prosthesis. Thus, in an exemplary embodiment, there is a method of evoking a hearing percept by electrically stimulating the cochlea based on a physical phenomenon within the cochlea, where, in at least some embodiments, the method is executed without intervening input from a component outside the recipient (i.e. no intervening input between the physical phenomenon within the cochlea and the stimulation of the cochlea). Alternatively, in an alternate exemplary embodiment, a signal indicative of the sensed physical phenomenon within the cochlea is outputted to an external component of the hearing prosthesis, which includes a sound processor, which sound processor processes the signal into a signal that is then transcutaneously transmitted to the receiver stimulator 180 inside the recipient where the receiver stimulator 180 utilizes that signal to output a signal to the electrode array of the electrode array assembly to electrically stimulate the cochlea and evoke a hearing percept. Additional details of such exemplary methods and systems and devices to execute such methods are detailed further below.

It is noted that while the embodiment of FIG. 3A has been disclosed with the sensor assembly 350 being an integrated, single unit with the electrode array assembly, in an alternate embodiment, the sensor assembly 350 is a separate unit from the electrode array assembly.

As noted above, the back volume 359 of the sensor assembly 350 includes a system which is initially indicated as black box 410. In an exemplary embodiment, black box 410 enables a static pressure difference between (i) an ambient environment (e.g., the static pressure in the cochlea of the recipient/the static pressure impinging upon the diaphragms 334) and/or a pressure in the front volume of the sensor (which is impacted by the ambient environment) and (ii) the back volume of the sensor and/or a combined front and back volume to be equalized, wherein both the back volume and the front volume are hermetically sealed/closed volumes relative to the ambient environment and, in some instances, relative to each other (in some embodiments as will be detailed below, the front and back volumes are in fluid communication with each other). In some embodiments, the sensor assembly itself is a single unit that enables one or more or all of the aforementioned static pressure equalization(s), while in other embodiments, the sensor assembly comprises two or more units, one or more of which enable one or more or all of the aforementioned static pressure equalization(s).

In this vein, FIG. 4 depicts a functional diagram of an exemplary sensor assembly having the functionality of sensor assembly 350 detailed above along with the aforementioned static pressure equalization functionality afforded by black box 410. Accordingly, FIG. 4 depicts a portion of an exemplary implantable device including an implantable sensor having a membrane 357 displaceable in response to a change in a physical phenomenon outside the sensor (e.g., a change in pressure of fluid inside a cochlea of a recipient due to ambient sound, as detailed above). (The implantable device can include the cochlear electrode array as detailed above, but in alternate embodiments, does not include the cochlear electrode array (e.g., it is only a sensor, not a stimulation device)). In this exemplary embodiment, the device is configured to equalize a static pressure difference between an ambient environment and a back volume of the sensor (which means that the device is configured to equalize a static pressure difference between an ambient environment and the front volume of the sensor in embodiments where the front volume in the back volume are in fluid communication with one another, at least when the fluid communication is such that a pressure change in the front volume relatively quickly causes a pressure change in the back volume). Accordingly, in an exemplary embodiment, the implantable device is configured to equalize a static pressure difference between an ambient environment and/or a front volume and a back volume of the sensor. In this exemplary embodiment, the expansion and/or contraction of the size of the back volume via black box 410 enables the equalization of the static pressure between the front volume and the back volume and/or between the back volume and the ambient environment and/or between the combined front and back volume and the ambient environment. More particularly, the implantable device is configured to adapt a volume of the back volume of the sensor and/or a combined front and back volume to a change in ambient pressure. In an exemplary embodiment, the implantable device includes a compliant back cavity that makes up at least a portion of the back volume.

Additional details of some embodiments will be described below, but first, some exemplary high-level functionalities will be described in view of the aforementioned functional schematic of FIG. 4.

As noted above, the fluid in the cochlea undergoes pressure variations caused by vibrations impinging upon the outside of the cochlea and transmission therein (e.g., through the oval window via ossicular vibrations (natural and/or prosthetically based), through the round window in scenarios where for whatever reason the round window transfers vibrations into the cochlea, and/or through any other part of the cochlea such that the cochlear fluid vibrates in a manner that the teachings detailed herein and/or variations thereof can be practiced). In at least some exemplary scenarios, the vibrations that impinge upon the outside of the cochlea and are transmitted therein are vibrations based on an ambient sound that would otherwise ultimately evoke a hearing percept in a normal hearing person. These vibrations cause pressure variations within the cochlea. This type of pressure variation results in what will be hereinafter referred to as dynamic pressure of the cochlea. It is this type of pressure variation (dynamic pressure) that the sensor assembly 350 detailed above and variations thereof sense to output a signal indicative of sound that can be utilized to evoke a hearing percept.

Conversely, pressure within the cochlea will change as a result of changes in the ambient environment, at least changes that are different than a change resulting from the phenomenon of sound. Hereinafter, the pressure within the cochlea resulting from such conditions is referred to as static pressure. Thus, dynamic pressure is a pressure relative to static pressure.

By way of example only and not by way of limitation, changes in atmospheric conditions in which a recipient of the sensor assembly 350 resides can result in a change in the pressure of the fluid inside the cochlea. One extreme exemplary example of this can occur when a recipient travels in a pressurized aircraft (e.g. a commercial jetliner having, for example, transatlantic capabilities, such as by way of example only and not by way of limitation, a Boeing 777 or an Airbus 380). It is routine for the cabin of the aircraft to be pressurized at an air pressure corresponding to the average air pressure at 8,000 feet above sea level. That is, the pressure inside the cabin is substantially lower than that which occurs at sea level. Over a sufficiently lengthy period of time (where lengthy is a relative term), the pressure inside the cochlea will equalize to, or at least reduce towards (at least in a significant manner that can impact the performance of the sensor assembly 350 as will be detailed below), the air pressure of the cabin. Another example of this can occur when a recipient swims underwater in general, and dives into the water in particular. That said, standard changes in atmospheric condition resulting from a passage of a low-pressure front or a high-pressure front (relative terms), ground travel resulting in altitude changes (common, for example, in the Western portions of North and South America) and other changes can also change the static pressure inside the cochlea. Moreover, in some instances, physiological changes of the recipient can result in changes in the static pressure of the front volume of the sensor assembly 350. By way of example only and not by way of limitation, in at least some embodiments, a hydration level of a recipient can potentially influence the static pressure within the cochlea.

Also it is noted that by static pressure changes, it is meant pressure changes that change relatively slowly. By way of example only and not by way of limitation, a pressure change resulting from a diver diving into a pool to a depth of 2 or 3 meters and then immediately ascending to the surface would not constitute a static pressure change. Conversely, if the diver were to remain at the depth of 2 or 3 meters for a period of time (a minute or more, for example, the change in ambient pressure would result in a static pressure change). In this regard, the affirmation scenario recognizes that in at least some embodiments implementing the teachings detailed herein and or variations thereof, a given equalization structure can require a lag time for pressure equalization. In an exemplary embodiment, this lag time is on the order of minutes, albeit in some embodiments the lag time is on the order of seconds.

Because the diaphragms 334 are deflected due to changes in pressure (both static and dynamic pressure), the aforementioned static pressure changes within the cochlea will influence the static pressure within the front volume of the sensor assembly 350, and within the combined front volume and back volume in embodiments where there is fluid communication between the two. Because the sensor 350 is configured such that dynamic pressure changes within the receptor 330 (e.g., resulting from sound) influence the membrane 357 of the microphone 354 (hence how the microphone 354 operates), static pressure changes within the receptor 330, and thus the front volume of the microphone 354, will cause the membrane 357 to be displaced from a neutral position.

That is, in at least some exemplary embodiments, the internal pressure of the front volume and/or back volume of the sensor assembly 350 is set to an initial internal pressure. In an exemplary embodiment, this is about 0.8 bars, which is average pressure at about 100 meters above sea level. The pressure can be set to be different depending on where the recipient spends most of his or her time (e.g., at sea level, in locations of heightened altitude, such as the city of Denver in the United States, which is about 1,200 meters above sea level, etc. that is the pressure is set to the average ambient atmospheric pressure). It is noted that in an exemplary embodiment, the internal pressure is set to a pressure that places the membrane 357 at a neutral position. In this regard, in an exemplary embodiment entails pressurizing or depressurizing the back volume to a pressure that places the membrane 357 at a neutral position for a specific ambient pressure.

It is noted that the teachings detailed herein and/or variations thereof can be practiced without the pressures in the front volume, the back volume and/or in the cochlea being equal. Embodiments can be practiced where there is an initial pressure difference, and this pressure difference is generally maintained during changes in the ambient environment so that the changes do not significantly impact the performance of the sensor assembly 350. Depending on the initial static pressure differential between the front volume and the back volume, a certain degree of deflection of the membrane 357 might result. In some embodiments, the deflection will be zero (e.g., where the front volume pressure and the back volume pressure are effectively equal). In other embodiments, the deflection will be nonzero (e.g., where the front volume pressure and the back volume pressure is not equal). Regardless of the initial deflection of the membrane 357, embodiments according to the teachings detailed herein and/or variations thereof reduce and/or eliminate the displacements of the diaphragm from its neutral position/deflection (whatever that may be) due to static pressure changes in the ambient environment. Indeed, some diaphragms 357 can have a natural memory that causes it to be bow shaped or the like even when pressures are equalized. Accordingly, embodiments detailed below will be described in terms of the membrane 357 relative to its neutral position, whether that be a zero deflection position or a nonzero deflection position.

As noted above, some embodiments are directed towards pressure equalization in a scenario where there is a combined front and back volume. In this regard, it is meant that there is fluid communication between the front and back volume. By way of example only and not by way of limitation, in an exemplary embodiment, the membrane 357 of the microphone can include one or more orifices (e.g., one or more piercings) that enables the flow of fluid from one side of the membrane 357 to the other side of the membrane 357, and thus from the front volume to the back volume, and vice versa. Accordingly, in an exemplary embodiment, the front volume and the back volume are not fluidically isolated from one another.

Unless otherwise explicitly stated herein, the teachings herein are applicable to embodiments where the front and back volumes are fluidically isolated from one another and embodiments where the front and back volumes are in fluid communication with one another (the latter being a combined front and back volume). Also unless otherwise stated herein, any phenomenon associated with the back volume as detailed herein can also corresponds to a phenomenon associated with the front volume, at least in embodiments where the front volume and back volume are in fluid communication with one another.

In this vein, most exemplary embodiments detailed herein are directed towards the embodiment where the front and back volumes are fluidically isolated from one another. However, it is noted that there is utilitarian value with respect to applying the teachings detailed herein to embodiments where the front and back volumes are in fluid communication with one another. In this regard, while the membrane 357 may not be deflected from the neutral position (or at least may not be significantly deflected from the neutral position) as a result of a difference in static pressure between the ambient environment and the combined front and back volumes, the diaphragms 334 may be deflected from their neutral positions. In this regard, it is noted that any teachings detailed herein associated with the membrane 357 can be applicable to the diaphragms 334. That is, for example, the diaphragms 334 can have neutral positions just as is the case with the membrane 357. In this regard, in scenarios where the static pressure of the ambient environment is greater than the static pressure within the front volume (and the static pressure within the combined front and back volumes in the case where there is fluid communication between the two volumes), the diaphragms 334 will be deflected inwards away from their neutral position. Conversely, in scenarios where the static pressure of the ambient environment is less than the static pressure within the front volume (and the static pressure within the combined front and back volumes in the case where there is fluid communication between the two volumes), the diaphragms 334 will be deflected outward away from their neutral position.

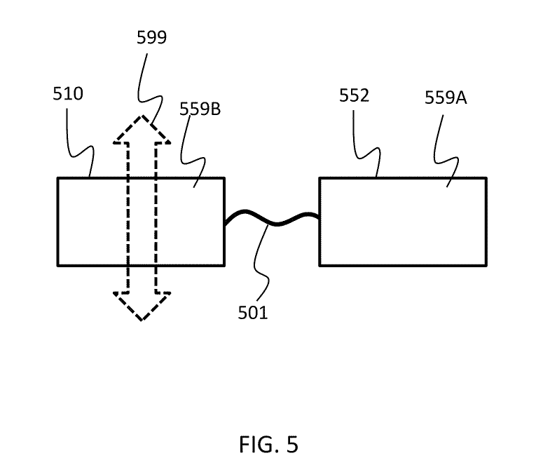

As noted above, an exemplary embodiment of the sensor assembly 350 utilizes device 410 to expand and/or contract the space constituting the back volume of the microphone 354. In an exemplary embodiment, the expansion and contraction is independent of movement of the membrane 357. FIG. 5 functionally depicts one exemplary embodiment where the back volume 359 is bifurcated into two sub-volumes 559A and 559B, where the two volumes are connected via tube 501, and thus the volumes are in fluid communication with one another.

In an exemplary embodiment, the tube 501 is a micro tube. Additional features of this micro tube will be described below.

More specifically, FIG. 5 functionally depicts an exemplary embodiment of the sensor assembly 350, where reference 552 corresponds to the housing depicted in FIG. 4 above, and reference 510 corresponds to an adaptive volume structure (corresponding to black box 410) remote from the housing 352, respectively encompassing sub-volumes 559A and 559B. Dashed arrow 599 represents the expandability and contractibility of the structure 510, and thus the volume 559B, and thus the back volume established by sub-volumes 559A, 559B and the volume of the inside of tube 501 (although in some embodiments, such is negligible with respect to the overall function of the sensor assembly).

Accordingly, in an exemplary embodiment, sensor assembly 350 includes a back volume that includes a first volume 559A and a second volume 559B remote from and distinct from the first volume 559A in fluid communication with the first volume 559A. When FIG. 5 is analyzed in view of FIG. 4, it will be seen that the first volume 559A is proximate the membrane 357 of the microphone 354 of the sensor assembly 350.

It is noted that the first volume 559A is located in a first housing/established by a first structure (housing 352 without the black box 510, where, instead, the black box 510 is replaced by a housing wall, as will be described in greater detail below) and the second volume is located in a second housing remote from the first housing, established by a second structure remote from the first structure and separable therefrom, where the second housing enables the expansion and contraction of the second volume.

Some exemplary features of the structures enabling the sensor assembly to have the functionality described above with respect to FIG. 5 will be described below, but first, an alternate embodiment will now be functionally described.

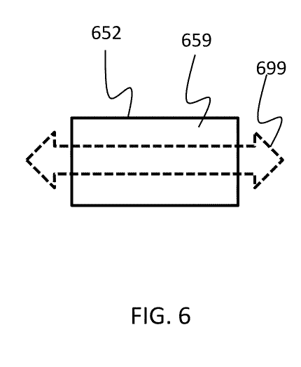

FIG. 6 functionally depicts another exemplary embodiment where the back volume is established by one single volume 659. More specifically, FIG. 6 functionally depicts an exemplary embodiment of a sensor assembly 350, where reference 652 corresponds to the housing 352 of FIG. 4 plus black box 410 depicted in FIG. 4 above, where the black box 410 represents an adaptive volume structure integrated into the housing 352. Dashed arrow 699 represents the expandability and contractibility of the structure 652, and thus the volume 659, and thus the back volume of the sensor assembly. Thus, in an exemplary embodiment, the back volume of the sensor assembly is established by a chamber bounded in part by the membrane 357, wherein the chamber is configured to vary the volume of the back volume in a manner beyond that resulting from displacement of the membrane 357. According to the embodiment of FIG. 6, the chamber is proximate the membrane 357. It is noted that in an exemplary embodiment, the expandability and contractibility of the structure 652 is independent of movement of the membrane 357.

As noted above, exemplary embodiments of the sensor assembly are such that the sensor assembly and a cochlear implant electrode array are part of a single unit. Accordingly, there is an exemplary embodiment that includes a sensor assembly including a compliant back cavity enclosure having the functionality as detailed herein and variations thereof integrated into a single unit (i.e., the electrode array assembly 390 is a combined electrode array 310 and the apparatus 320 including the compliant back cavity) with a cochlear implant electrode array. This is as differentiated from, for example, a sensor assembly according to the embodiment of FIG. 5, where adaptive volume structure 510 is remote from the housing 352, and connected thereto by tube 501 or otherwise merely attached to the remainder of the sensor assembly in a non-unitized manner. Thus, the adaptive volume structure 510 is part of a separate unit that is separate from the unit of the electrode array 310/housing 352.

In view of the above, it is noted that embodiments based on the functional schematics of FIGS. 5 and 6 utilize expansion of the volume of the back volume in response to a decrease in static pressure on an opposite side of the membrane 357 (in the front volume) relative to the back volume and/or in response to a decrease in the static pressure in the ambient environment relative to the combined front and back volume, thereby equalizing the pressures between the front volume and the back volume (irrespective of movement of the membrane 357) and/or between the combined front and back volume in the ambient environment (irrespective of movement of the diaphragm(s) 334). Also in view of the above, it is further noted that embodiments based on the functional schematics of FIGS. 5 and 6 utilize contraction of the volume of the back volume in response to an increase in static pressure on the opposite side of the diaphragm (in the front volume) relative to the back volume and/or in response to an increase in the static pressure in the ambient environment relative to the combined front and back volume, thereby equalizing the pressures between the front volume and the back volume (irrespective of movement of the membrane 357) and/or equalizing the pressures between the combined front and back volume and the ambient environment (irrespective of movement of the diaphragm 334). Thus, embodiments include a device, such as a hearing prosthesis, that is configured such that expansion and contraction of the volume of the back volume equalizes the static pressure on the opposite side of the membrane with the static pressure in the back volume, irrespective of movement of the membrane 357. Still further, embodiments include a device, such as a hearing prosthesis, that is configured such that expansion and contraction of the volume of the back volume equalizes the static pressure in a combined front and back volume with that on the opposite side of the diaphragms 334 (e.g., inside the cochlea, which can correspond to the ambient environment), irrespective of movement of the diaphragm 334.

Some more specific features of the embodiment of FIG. 5 will now be described, followed by more specific features of the embodiment of FIG. 6.

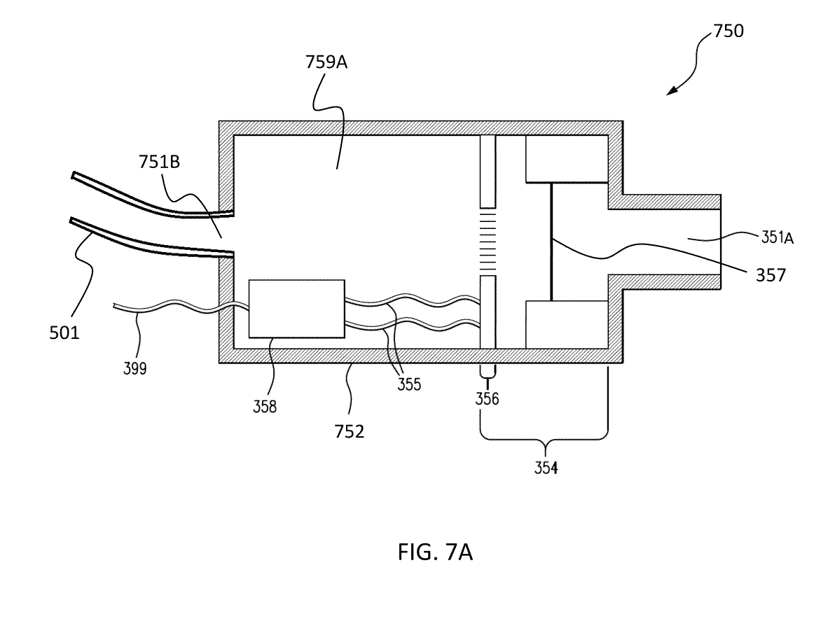

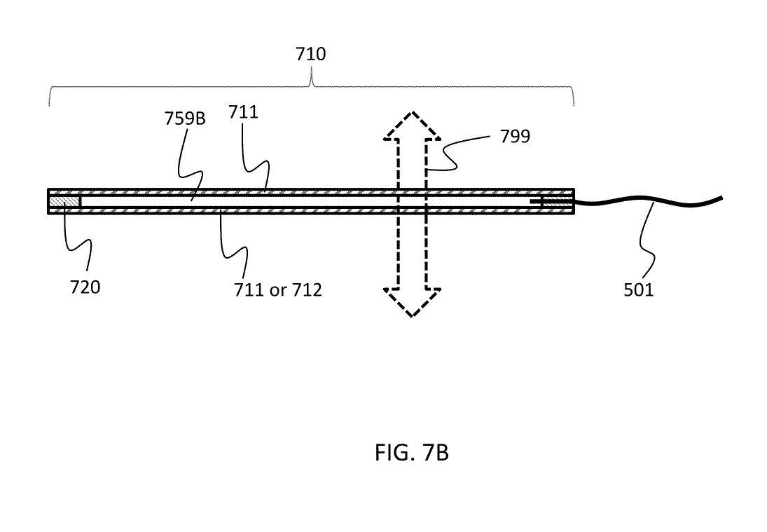

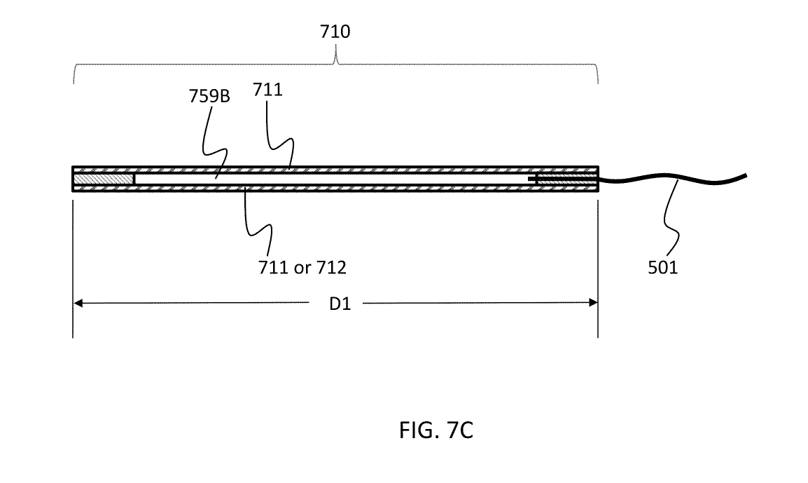

FIG. 7A depicts a cross-sectional view of a portion of an exemplary sensor assembly 750 that corresponds to sensor assembly 350 of FIG. 4. As can be seen, the sensor assembly 750 includes housing 752 that has two ports 351A and 751B. Port 751B opens volume 759A to tube 501. FIG. 7B depicts a schematic of adaptive volume structure 710 that is also a part of sensor assembly 750. It is noted that the embodiment of the adaptive volume structure 710 in FIG. 7B is merely exemplary and presented in quasi-functional terms. As will be detailed below, additional structure can be utilized in the adaptive volume structure 710 to enhance or otherwise provide utilitarian value with respect to long-term implantation in a recipient.

Common to both FIGS. 7A and 7B is tube 501. Accordingly, tube 501 connects the housing 752, or more particularly, the interior volume 759A (the volume inside the housing 752 to the left of membrane 357 the back volume in the housing 752), to the interior volume 759B of adaptive volume structure 710. Like reference numbers of FIG. 7A correspond to like reference numbers of FIG. 4 (housing 752 corresponding to housing 352 save for the addition of the port 751B). Accordingly, elements 501, 751B, 759A and the elements of FIG. 7B make up the components of the black box 410 of FIG. 4 and have the functionality thereof. Also, with reference to FIG. 5, reference 552 corresponds to the housing 752 depicted in FIG. 7A, and reference 510 corresponds to the adaptive volume structure 710 of FIG. 7B. Sub-volumes 559A and 559B of FIG. 5 correspond to sub-volumes 759A and 759B, respectively.

As will be detailed further below, adaptive volume structure 710 includes one or more diaphragms 711. The diaphragm(s) are configured to flex/stretch inward and/or outward, as functionally represented by arrow 799, thereby varying the size of the volume 759B. Accordingly, dashed arrow 799 corresponds to dashed arrow 599, and likewise represents the expandability and contractibility of the structure 710, and thus the volume 759B, and thus the back volume established by sub-volumes 759A, 759B and the volume of the inside of tube 501.

Some structural features of the adaptive volume structure 710 of FIG. 7B will now be described. As can be seen, in a basic form, the adaptive volume structure 710 includes a spacer ring 720 (a top view of the structure 710 (i.e., looking in the vertical direction of the plane of FIG. 7B) would reveal that the structure 710 has a circular outer periphery although in other embodiments, it can have a periphery of an alternative configuration) to which is connected two diaphragms 711. In an exemplary embodiment, the diaphragms 711 are clamped to the ring 720. In an exemplary embodiment the diaphragms are directly bonded (via welding, adhesives, etc.) to the ring 720. In an exemplary embodiment, the ring is made out of titanium (including titanium alloys). Indeed, in an exemplary embodiment, every structural component of the adaptive volume structure 710 (as well as at least some adaptive volume structures detailed herein) is made out of titanium (disclosure of titanium herein includes titanium alloys). Accordingly, this can provide a biocompatible and hermetic sensor structure

By way of example only and not by way of limitation, the diaphragms correspond to diaphragms manufactured via standard photolithography and dry etching processes. In at least some embodiments, the titanium diaphragms 711 are titanium foils. The titanium diaphragms have thickness of about 10 micrometers, although thicker and/or thinner diaphragms can be utilized (e.g., thicknesses of about 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, and/or about 20 μm or more or less or any value or range of values therebetween in about 1/10^{th} micrometer increments (e.g., 8.3 micrometers, 12.1 micrometers, 6.6 micrometers to about 18 micrometers, etc.). In an exemplary embodiment, the titanium diaphragms are manufactured from thin wafers due to the fact that the titanium exhibits relatively high fracture toughness.

In an exemplary embodiment, the diaphragms 711 are corrugated diaphragms having a thickness of about 12 micrometers. In an alternate embodiment, the diaphragms are flat diaphragms having a thickness of about 10 micrometers

It is further noted that in at least some embodiments, the thicknesses of the diaphragms are relatively constant. That said, in an alternative embodiment, the thicknesses of the diaphragms vary with distance along the diameter. By way of example only and not by way of limitation, the thicknesses of the diaphragms located at or proximate to the rings can be thicker than the thicknesses of the diaphragms located away from the rings (i.e. the portions that flex). Indeed, in at least some embodiments, the rings can be dispensed with—the diaphragms being monolithic components with components that have the functionality of rings. Still further by way of example only and not by way of limitation, in at least some embodiments, the diaphragms can have raceways that are relatively thin relative to the remainder of the diaphragms. That is, in an exemplary embodiment, the diaphragms can have path(s) that circumnavigate a geometric center of the diaphragms of relative thinness located on the outer locations of the diaphragm but inboard of the rings. It is these locations that provide most of the flexure, or at least the greatest local degree of flexure, with the remainder of the diaphragms being relatively inflexible.

Referring to FIG. 7C, it is noted that the diameter D1 of the diaphragms 711 is about 19 mm, and the diameters of the ring 720 can be considered about drawn to scale. In an exemplary embodiment, the diameter D1 is about 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, or more (or less), or any value or range of values therebetween in about 1/10 of a millimeter increment. In an exemplary embodiment, the ring 720 is in contact with the diaphragm(s) over about ½ of the diameter of the diaphragms. In an exemplary embodiment, the ring 720 is in contact with the diaphragm(s) over about 1/10^{th}, 1/9^{th}, ⅛^{th}, 1/7^{th}, ⅙^{th}, ⅕^{th}, ¼^{th}, ⅓^{rd}, ½, 6/10 ths or 7/10 ths or more or less of the diameter of the diaphragms or any value or range of values in about 1/100 ths of a diameter increments. In an exemplary embodiment, the unclamped diameter of the diaphragms 711 is about 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm or any value or range of values therebetween in 0.1 mm increments.

Any configuration of the diaphragm-ring assembly that can enable the teachings detailed herein and are variations thereof to be practiced can utilize in at least some embodiments.

As can be seen, tube 501 extends through one side of the ring 720 into the interior volume 759B, thus placing that volume into fluid communication with volume 759A of the housing 752. While tube 501 is depicted as passing through the ring 720, the tube can instead stop short of the extension into the volume 759B depicted in FIG. 7B. Indeed, it could instead connect to a port of the ring 720, where a bore extends through ring 720 to the volume 759B. Any device, system or method that will enable tube 501 to place volume 759B into fluid communication with volume 759A can be used in at least some embodiments.

Thus, adaptive volume structure 710 includes a stack of clamped diaphragms 711, wherein the diaphragms 711 are configured to deflect in first directions and second directions (inward into volume 759B and outward away from volume 759B), thereby respectively contracting and expanding the back volume (volume 759A plus volume 759B plus the volume of the inside of the tube 501) independent of the movement of the membrane 357.

Still with reference to FIG. 7B, an alternate embodiment can include a rigid component 712 instead of a diaphragm 711 at one location. That is, instead of having two diaphragms 711, the adaptive volume structure 710 can include only one diaphragm. As will be detailed below, some embodiments include more than two diaphragms. Any number of diaphragms that will enable the teachings detailed herein and/or variations thereof to be practiced can be utilized in at least some embodiments.

Thus, as can be seen from FIGS. 7A and 7B, there is an exemplary pressure equalization system that includes two separate units distinct from one another housing 752 and adaptive volume structure 710. The microphone is part of the first unit and the second unit is configured to expand and contract (either by deflection of one or two diaphragms 711) such that the volume of the back volume is expanded and contracted (via expansion and contraction of volume 759B) independent of movement of the membrane 357, where tube 501 places the two units into fluid communication with one another.

In an exemplary embodiment, the adaptive volume structure 710 is implanted in the recipient beneath the outer layer of the skin of the recipient at a location such that the diaphragm(s) 711 are deflected dependent on a difference between the ambient pressure relative to the location of the receptor 330 and the internal pressure (back volume and/or combined front and back volume), thereby modifying the size of the back volume of the microphone and returning and/or maintaining the membrane 357 at a neutral position (and/or the diaphragm(s) 334 at the neutral position). In at least some exemplary embodiments, the adaptive volume structure 710 is located above the mastoid bone of the recipient (e.g., behind and/or above the ear canal of the recipient). In an exemplary embodiment, it is configured to be located between the outer surface of the mastoid bone and the skin of recipients.

Accordingly, in an exemplary embodiment, diaphragm(s) numeral 711 are exposed to the ambient environment, and thus the ambient pressure at a location between the mastoid bone and the outer surface of the skin of the recipient. Thus, pressure changes in the ambient environment will cause the diaphragm(s) 711 to defect, thereby varying the volume 759B, and thus equalizing the pressure between the front volume and the back volume (or between the ambient environment and the combined front and back volume), because the pressure of the ambient environment proximate the surface(s) of the diaphragm(s) 711 will be substantially about the same as the pressure of the environment within the cochlea where receptor 330 is located (which influences the pressure of the front volume). Thus, the deflection of the diaphragm(s) 711 will vary the interior volume 759B, and thus equalize the pressures between the back volume and the front volume of the microphone of the sensor 350 (and/or between the combined front and back volume and the ambient environment).

As noted above, embodiments of the adaptive volume structure 710 can use one or two diaphragms. Embodiments that utilize one diaphragm where instead of two diaphragms, one rigid plate 712 is utilized in place of the diaphragm can have utilitarian value where the flexation/stretching of that one diaphragm 711 is sufficient to enable the teachings detailed herein and/or variations thereof, such as to equalize the pressures between the front and back volume and/or between the total combined volume and the ambient environment, where the rigid plate 712 provides protection to the adaptive volume structure.

In an exemplary embodiment, the back volume of the sensor 750 (the volume “to the left” of membrane 357—volume 759A, volume 759B and the internal volume of tube 501), which is a variable volume owing to the diaphragm(s) 710, is significantly larger than the front volume (volume “to the right” of membrane 357—the internal volume of the receptor 330, the internal volume of tube 340 and the portion of the sensor 350 inside housing 752 not including portion 359 (with reference to FIG. 4). In an exemplary embodiment, the size of the back volume is about 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more times the size of the front volume. Any ratio of volumes of the back volume, which is a variable volume, to the front volume, which is a constant volume (or at least and effectively constant volume in that the movement of the diaphragm is negligible relative to changing the volume of the front volume) that can enable the teachings detailed herein and are variations thereof to be practiced can utilize in at least some embodiments. Along these lines, additional features of the front and back volume relationship will be described below, but first, some alternate embodiments of alternate adaptive instructions will now be described.

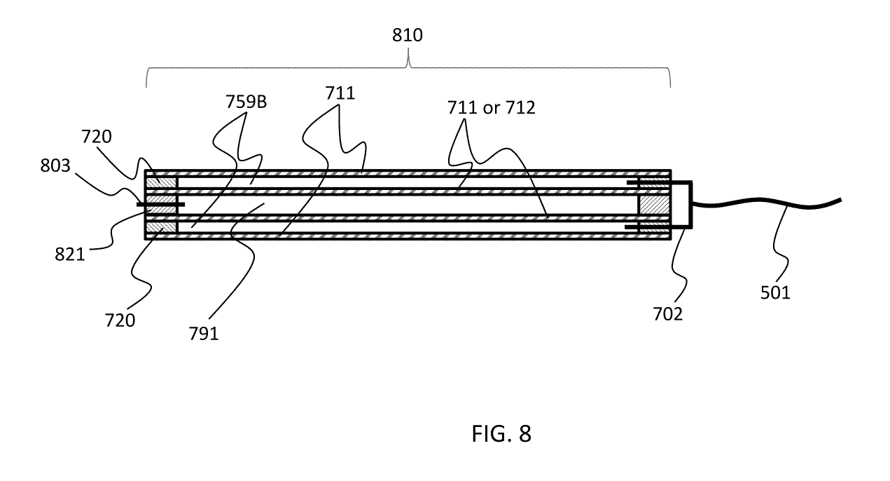

At least some embodiments utilize a plurality of volumes 759B that are manifolded together, and thus pneumatically interconnected. In this regard, FIG. 8 presents an alternative embodiment of an adaptive volume structure 810. In an exemplary embodiment, adaptive volume structure 810 corresponds to a duplication of adaptive volume structures 710, one on top of the other, separated by a ring 821, as can be seen. In an exemplary embodiment, all the components are clamped together. Ring 821 establishes a volume 791 between the two assemblies corresponding to adaptive volume structures 710 (i.e., between diaphragms 711 or rigid plates 712). Accordingly, embodiments utilizing for diaphragms 711 constitute an adaptive volume structure that utilizes two pairs of volume adapting diaphragms. As can be seen, the volumes 759B, and thus the diaphragms 711/plates 712 are arranged in a stack. In embodiments utilizing four diaphragms 711, the volume 791 is vented or otherwise placed into fluid communication with the ambient environment (the environment between the bone and the outside surface of the skin of the recipient) of the adaptive volume structure 810. In an exemplary embodiment, this is achieved via a conduit through ring 821. In the exemplary embodiment depicted in FIG. 8, a tube 803 is utilized, as can be seen. Accordingly, the pressure of volume 791 is about the same as (including the same as) the ambient pressure on the outside of the adaptive volume structure 810 (i.e., the pressure impinging upon the surfaces of the outer diaphragms 711). Any device system or method that can enable fluid communication from the outside of the adaptive volume structure 810 to volume 791 can be utilized in at least some embodiments provided that the teachings detailed herein and are variations thereof can be executed.

Fluid communication between the ambient environment and volume 791 is utilitarian for embodiments where four diaphragms 711 are utilized. In this regard, the ambient pressure is exposed not only to the diaphragms 711 on the outside of the adaptive volume structure 810 (i.e., the top and bottom diaphragms), but also to the diaphragms located in the middle of the adaptive volume structure 810. That said, in an alternative embodiment, where rigid plates alike are utilized for the middle components, it may not be necessary to have fluid communication between volume 791 and the ambient environment. Indeed, in such embodiments, volume 791 may not exist. Instead, the rigid plates can be located back to back without a volume therebetween, or, in an alternative embodiment, a single rigid plate can be utilized; one side of the plate establishing one of the volumes 759B and the other side of the plate establishing the other of the volumes 759B—both of the volumes 759B being variable volumes owing to the fact that each is bounded by a diaphragm 711 that has a surface exposed to the ambient environment. Further, in alternate embodiments, the rigid plates can be located on the outside surfaces of the adaptive volume structure 810. That is, flexible diaphragms can be utilized for the middle to diaphragms, which will be exposed to the ambient pressures via tube 803, and thus will flex with pressure changes, thus causing the volumes 759B to vary.

As noted above, volumes 759B are manifolded together. As can be seen in FIG. 8, tube 501 leads to manifold 702. Thus, utilitarian value of varying both of the volumes 759B can be harnessed in that the variations of both of the volumes 759B can be used to equalize the pressure of the back volume to that of the front volume and/or the pressure of the combined front and back volume to that of the ambient environment. In this regard, the amount that the volumes vary is effectively doubled, all other parameters being equal (which they may not be in embodiments where different diaphragm configurations (thickness, diameter, smooth vs. corrugated, etc.) are utilized, as further detailed below).

It is again reiterated that the FIGS. 7A-8 are quasi-functional figures and that the actual implemented embodiments may not necessarily correspond to the configurations depicted therein. Along these lines, it can be seen that tube 803 juts outward away from the outer periphery of the adaptive volume structure 810. In an exemplary embodiment, tube 803 may end at a location flush with and/or recessed with the outer surface of the ring 821, or tube 803 may not be present at all a bore through ring 821 may instead be present. Also, a filtering system or the like may be located at the entrance of the tube 803 to filter out at least some body fluids and/or tissue, thereby preventing or at least limiting the ingress of tissue and/or at least some body fluids into volume 791. Additional features of such a “filter” are described below. Further, while the manifold 702 is depicted on the outside of the adaptive volume structure 710, an exemplary embodiment can be such that the tube 501 enters a port in the ring 821. Ring 821 can include a passage that extends from the port in the vertical direction (upwards and downwards relative to the frame of reference presented by FIG. 8) through the middle of the ring body, and then through the diaphragms (or rigid plates as the case may be) and then into rings 720 and then dogleg to ports located in ring 720 on the inside thereof, thus placing volumes 759B into fluid communication with each other and with tube 501 utilizing a manifold system that is completely internal to the adaptive volume structure 810. This alternate manifold structure can be achieved utilizing bores through the various components that are made therein prior to assembly of the components—the bores being aligned with each other to create passageways through the structure to the inside of the adaptive volume structure 810. Functionally, this can correspond to moving manifold 702 and tube 501 to the left, relative to the frame of reference presented by FIG. 8, such that the manifold is located entirely within the rings, diaphragms, and plates of the adaptive volume structure 810. As with the tube 803, instead of tubing as depicted in FIG. 8, bores through the components can be utilized.

Any device, system, and/or method that can place the pertinent volumes into fluid communication with one another to enable the teachings detailed herein and/or variations thereof can be utilized in at least some embodiments.

In view of FIG. 8, it can be seen that an exemplary embodiment includes a back volume that includes a first sub-volume (upper volume 759B) bounded by at least a first diaphragm (any of the top two diaphragms 711), and a second sub-volume (lower volume 759B) bounded by at least a second diaphragm (any of the bottom two diaphragms 711). In at least some exemplary embodiments, the first sub-volume is in fluid communication with the second sub-volume (e.g., by manifold 702 or whatever other conduit system and/or manifold system that can enable the teachings detailed herein and are variations thereof to be practiced). The sub-volumes are arrayed in the direction normal to the maximum diameter of at least one of the diaphragms forming at least one of the aforementioned boundaries. Further, a first size of the first sub-volume is independent of a second size of the second sub-volume. In this regard, in embodiments utilizing identical diaphragms and/or identical rigid plates as the case may be, the sizes of the volumes 759B can differ based on the thickness/height of the rings 720, etc. Alternatively and/or in addition to this, in an alternate embodiment, the diaphragms can have different diameters. By way of example only and not by way of limitation, in at least some embodiments, the rings can be partial cones such that an outer diameter thereof at one end is larger than the outer diameter at the other end, thus permitting a larger un-clamped diameter of a given diaphragm. Alternatively and/or in addition to this, the rings can be configured such that they impart a slope onto a diaphragm relative to another diaphragm. Any device, system, and/or method of establishing independence between a given volume/sub-volume can be utilized in at least some embodiments providing the teachings detailed herein and variations thereof can be practiced.

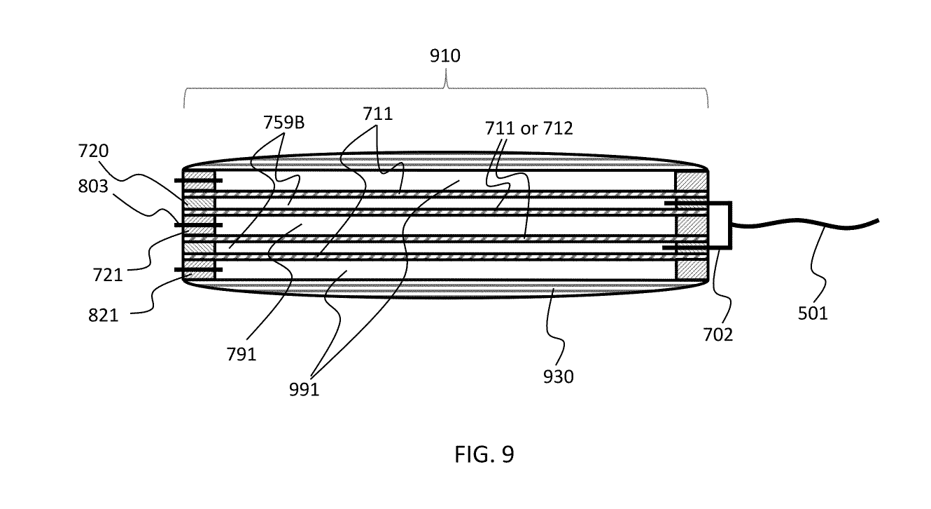

FIG. 9 depicts yet another alternate embodiment of an adaptive volume structure 910. Adaptive volume structure 910 corresponds to the adaptive volume structure 810 of FIG. 8, with the addition of two additional rings 821 respectively on the bottom and top thereof, plus respective caps 930 attached to the additional rings. In an exemplary embodiment, these are rigid caps configured to protect the outer diaphragms 711. As can be seen, each of the rings 821 include tubes 803 extending therethrough configured to place the volumes 991 established by the caps 930 and the outer diaphragms 711 into fluid communication with the ambient environment in a manner concomitant with the tube 803 of FIG. 8 vis-à-vis volume 791. Accordingly, as can be seen, every diaphragm is exposed to the pressure of the ambient environment even though rigid caps 930 are interposed between the ambient environment and the diaphragms. Thus, any change in ambient pressure that would result in deflection of the diaphragms of the embodiment of FIG. 8 still results in deflections of the diaphragms of the embodiment of FIG. 9 in a manner that is at least about the same as (including the same as) that which occurs in the embodiment of FIG. 8. Accordingly, an exemplary embodiment includes a stack that includes one or more diaphragms, one or more substantially rigid components (plates and/or caps), and one or more spacers spacing apart two diaphragm or a rigid component. In an exemplary embodiment, the stack of clamped diaphragms of FIG. 8 is about 1 millimeter in height.

It is noted that as with the embodiments of FIGS. 7A-8, the embodiment of FIG. 9 is presented in a quasi-functional format. As was detailed above in the embodiment of FIG. 8, manifold 702 may not be as pronounced as that depicted in FIG. 9. Further, tubes 803 may not necessarily be present. As with the manifold of the embodiment of FIG. 8, fluid communication between volumes 791 and 991 and the ambient environment may be achieved in an analogous manner. By way of example only and not by way of limitation, one or more ports may be located on the outside of one or more rings 821, which lead to vertical bores through the various components which then dogleg towards the interior of the adaptive volume structure 910 to place volumes 991 into fluid communication with the ambient environment (a bore can extend from the outside directly to the inside through middle ring 821 placing volume 791 into fluid communication with the ambient environment and/or into fluid communication with one or both volumes 991). Moreover, while the embodiment of FIG. 9 presents only a single passage through each of the rings 821, embodiments can utilize two or more passages through any given ring 821, with an internal manifold system connecting those passages to the volumes 791 and/or 991. Such can also be the case with respect to placing to 501 into fluid communication with the volumes 759B.

It is noted that alternatively and/or in addition to the rings 821, the caps 930 can be configured such that they have hollow portions therein that provide a space to establish volumes 991. By way of example only and not by way of limitation, in at least some embodiments, rings 821 can be monolithic components with caps 930. Indeed, in an exemplary manufacturing process, cap 930 is machined to place a circular hollow portion therein to provide for the volume 991 when a diaphragm 711 is attached to cap 930.

Also, while vertical and horizontal bores have been referenced above, where it has been implied that the directions of the bores are linear, curved bores can be utilized as well. By way of example only and not by way of limitation, in at least some embodiments, curved conduits can be machined or otherwise formed into the upper and/or lower portions of the rings and/or the rings can be bifurcated, at least partially, into outer rings and inner rings, where fluid conduits are located between the outer rings and inner rings. Such can be achieved via manufacturing processes where each ring and each diaphragm and each cap is a separate component that is ultimately stacked up and connected to each other during assembly, where there is easy access to any side of any individual component prior to assembly. Again, any device, system and/or method that can enable fluid communication between the various volumes and/or the ambient environment can be utilized in at least some embodiments.

In view of the above, it is now noted that an exemplary embodiment includes an implantable static pressure equalization system configured to equalize an internal pressure of an apparatus, such as the sensor assembly 350, that is configured to sense a dynamic phenomenon in a recipient (e.g., such energy travelling through the fluid of the cochlea resulting from ambient sound) with a static pressure of an ambient environment. As can be seen from FIGS. 7A-8, the system includes at least one diaphragm 711 bounding a volume (e.g., the back volume). The diaphragm 711 is configured to deflect in response to a change in the static pressure, thereby adjusting the size of the volume bounded by the diaphragm (i.e., volume 759B, which is part of the back volume). The system is configured such that the volume is placed in fluid communication with the apparatus, such as via tube 501 (with or without manifold 702). With respect to the embodiment of FIG. 9, the diaphragm(s) are sheltered by at least two substantially rigid components (caps 930) located on opposite sides of the diaphragms in a direction normal to a maximum diameter of the diaphragms.

Further, still with reference to FIG. 9, as noted above, tube 803 extends into the ambient environment. In at least some embodiments, tube 803 enables ingress and egress of a body fluid between the diaphragm(s) 711 bounding volume 791. Conversely, the adaptive volume structure 810 (or 710) is configured such that the volume in fluid communication with the microphone of sensor 350 (the back volume), such as variable volume 759B, is hermetically sealed from the body fluid when the volume (the back volume) is placed in fluid communication with the microphone. Accordingly, owing to the passageways provided by tubes 803 (or whatever manifold or conduit system that is utilized in a given embodiment), the adaptive volume structures can include non-hermetic volume(s) 791 and/or 991 that are hermetically isolated from volumes 759B, and thus the back volume. These non-hermetic volume(s) extend in between at least one of the substantially rigid components 930 and at least one of the diaphragms 711. As will now be detailed in an exemplary embodiment, one or more or all of these non-hermetic volume(s) are separated from the ambient environment by silicone housing.

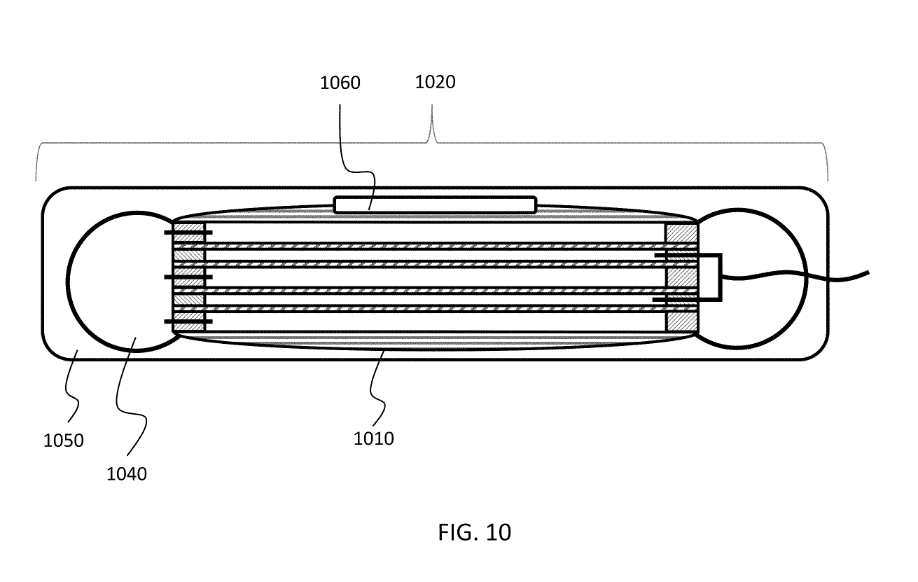

In an exemplary embodiment, silicone housing encompasses the stack of the diaphragms, the caps and the spacer (e.g., adaptive volume structure 910). More specifically, with reference to FIG. 10, an assembly 1020 is presented established by an adaptive volume structure 1010 encased in a silicone housing 1050. Adaptive volume structure 1010 corresponds to adaptive volume structure 910 of FIG. 9 with the inclusion of a ferromagnetic material component 1060, which in an exemplary embodiment is a permanent magnet (additional details of which are described below).

Briefly noted above is the concept of a “filter” to prevent or otherwise limit tissue ingress into volumes 791 and/or 991, which are in fluid communication with the ambient environment via tubes 803 (or whatever other mechanism is used for fluid communication). Along these lines, silicone housing 1050 forms an open volume 1040 which is generally donut shaped that circumnavigates the outer periphery of the adaptive volume structure 1010, although in alternate embodiments, it need not circumnavigate the adaptive volume structure 1010—any configuration or extension of the volume that can enable the teachings detailed herein that are variations thereof to be practiced can be utilized in at least some embodiments. This open volume is in fluid communication with the volumes 791 and 991. Accordingly, in this exemplary embodiment, volume 1040 is an integral part of the silicone structure which houses the adaptive volume structure 1010 and forms another adaptive volume. In this regard, pressure changes in the ambient environment in which the assembly 1020 is located (e.g., the environment between the mastoid bone and the surface of the skin of the recipient, etc.) results in expansion or contraction of the size of the volume 1040, thereby at least effectively equalizing the pressure of the volume 1040 with the ambient environment. Because the volumes 791 and 991 are in fluid communication with the volume 1040, pressure changes in the volume 1040 are communicated to the volumes 791 and 991. These pressure changes in turn result in deflections of the diaphragms as detailed above, and thus changes in the volumes 759B as detailed above.

In an exemplary embodiment, by way of example only and not by way of limitation, the silicone of the housing 1050 is relatively highly elastic, and the structure of the housing 1050 is such that the portions of the housing that create the volume 1040 results in a sufficiently elastic structure that enables the volume 1040 to be an adaptive volume, in a manner concomitant with the adaptive volume of the back volume of the microphone of sensor 350. In this regard, an exemplary embodiments includes a sensor according to any of the sensors detailed herein having a microphone having a first back volume and a second back volume, where the first back volume is fluidically isolated from the second back volume. In an exemplary embodiment, both the first back volume and the second back volume are adaptive back volumes. In the embodiment of FIG. 10, the first back volume is located in series with the second back volume.

In an exemplary embodiment, the silicone of the housing 1050 provides protection against contamination of volumes 791 and 991 with human tissue. That is, volume 1040 is not a hermetically sealed volume, and thus volumes 791 and 991 are likewise not hermetically sealed volumes.

As noted above, embodiments of the sensor 750 are configured to sense a physical phenomenon within the cochlea of a recipient, and the adaptive volume structures associated therewith are configured to be located between the mastoid bone and the outer surface of the skin in back of and/or above the ear canal of the recipient. Accordingly, in an exemplary embodiment, the tube 501 is configured to extend from the housing 752 of the sensor 750, which is located proximate to the cochlea as can be seen in FIG. 3B, to the location of the adaptive volume structure 710 just noted. In an exemplary embodiment, the length of the tube 501 is about 90 mm. In an exemplary embodiment, the adaptive volume structures detailed herein and variations thereof are configured for use with a cochlear implant, such as the cochlear implants of FIGS. 1A-1B detailed above. Indeed, as will now be described by way of example, an exemplary embodiment includes an adaptive volume structure according to any of the embodiments detailed above that is fully integrated into a cochlear implant. The following is a description of such an embodiment with reference to utilization of the assembly 1020 of FIG. 10 in a cochlear implant.

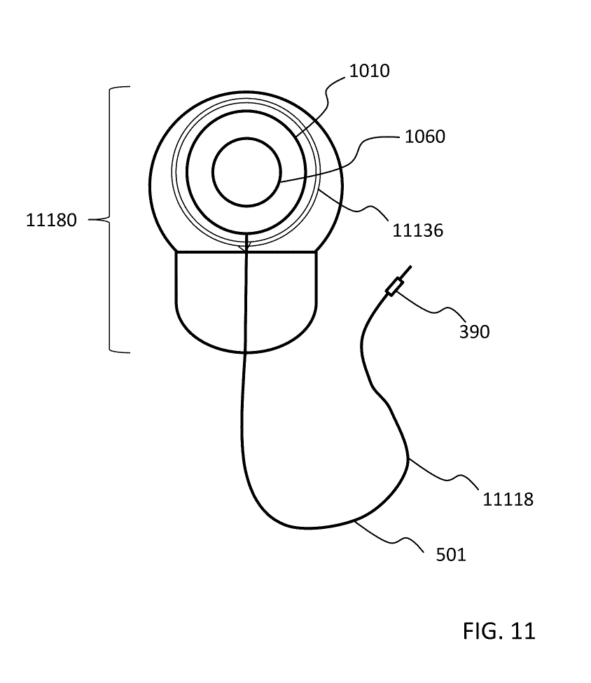

More specifically, FIG. 11 depicts an exemplary internal component of a cochlear implant system, corresponding to internal component of FIG. 1B, which corresponds to the internal component of FIG. 1A, both of which are detailed above. As can be seen, the internal component includes a receiver simulator 11180 corresponding to receiver simulator 180 of FIG. 1B, with the inclusion of adaptive volume structure 1010 thereto, around which antenna coil 11136, corresponding to primary internal coil 136 detailed above, extends.

From the receiver stimulator 11180 there extends an elongate stimulating assembly 11118 corresponding to the elongate stimulating assembly 118 detailed above which includes electrode array assembly 390. The elongate stimulating assembly 11118 includes and/or runs parallel to tube 501 (in an exemplary embodiment, the tube 501 is integral with the other components of the elongate stimulating assembly 118). In an exemplary embodiment, the tube 501 is integrated into the structure of the stimulator of the internal component. In an exemplary embodiment, the tube 501 can run directly through the stimulator or run around the periphery (side, above, etc.) of the stimulator component to reach the adaptive volume structure 1010. In an exemplary embodiment, the tube 501 can connect to a component of the stimulator, and thus the stimulator can place the microphone into fluid communication with the adaptive volumes of the adaptive volume structure 1010 (another tube or some other component can place the adaptive volume structure 1010 into fluid communication with the stimulator). In an exemplary embodiment, electrical leads extending between the elongate stimulating assembly 390 and the receiver-stimulator 11180 are located in the tube 501 (i.e., inside the conduit established by tube 501).

Consistent with other internal components of cochlear implants, the receiver stimulator 11180 is encapsulated in silicone. Accordingly, the adaptive volume structure 1010 is also encapsulated in silicone. In an exemplary embodiment, the encapsulation is such that an adaptive volume corresponding to volume 1040 is present therein. Indeed, in an exemplary embodiment, the receiver stimulator 11180 corresponds to a combination of assembly 1020 of FIG. 10 with the inclusion of wire antennas 11136 in the housing 1050 circumnavigating or running along with volume 1040, where the housing 1050 extends to encapsulate the simulator portion. That is, in an exemplary embodiment, receiver simulator 11180 further includes volume 1040, which can be interposed between adaptive volume structure 1010 and antennas 11136.

Also consistent with other internal components of cochlear implants, the elongate stimulating assembly 118 is also encapsulated in silicone, at least to the point of the electrodes thereof. With respect to the latter, the tube 501 and the leads extending from the electrode array assembly 390 can be encapsulated in the same silicone.

It is noted that in this exemplary embodiment, electrode array assembly 390 utilizes the sensor assembly 750 detailed above.

As can be seen from FIG. 11, ferromagnetic structure 1060 (e.g., permanent magnet) is located at about the traditional location where such magnets are located in traditional cochlear implants. Accordingly, an embodiments where the adaptive volume structure is fully integrated into a cochlear implant can have utilitarian value in that the ferromagnetic structure 1060 can be utilized to establish magnetic attraction between the external component and the internal component of the cochlear implant system and/or can be utilized to center the external coil relative to the internal coil, thereby enhancing communication between the two components. It is noted while the embodiment of FIG. 10 depicts a ferromagnetic component 1060 fully integrated into the adaptive volume structure 1010, an alternative embodiment, the ferromagnetic component 1060 is a separate component from the adaptive volume structure 1010 (e.g., the component 1060 can be encapsulated in silicone with but separate from the adaptive volume structure 1010, and can be located on or spaced away from the adaptive volume structure 1010.

Accordingly, from the above, it can be seen that in an exemplary embodiment, the adaptive volume structure 1010 comprises a stack of diaphragms 711, caps 930, spacers 720, 721 and 821 and a ferromagnetic component 1060, such as a permanent magnet, along with a receiver coil 11136 of a transcutaneous electromagnetic communication system, all of which are encompassed in a silicone housing 1050. Further, from the above, it can be seen that an exemplary embodiment includes a cochlear implant including a receiver-stimulator component, a cochlear implant electrode array 390 including a microphone configured to be located proximate to and/or in the cochlea of the recipient, and an adaptive volume structure according to any of the embodiments detailed herein and/or variations thereof, wherein a volume of the back volume extends from the electrode array of the cochlear implant to the receiver-stimulator component 11180.

As noted above, FIG. 6 presents an alternate embodiment relative to that of FIG. 5. Now, some specific features of the embodiment of FIG. 6 will now be described.

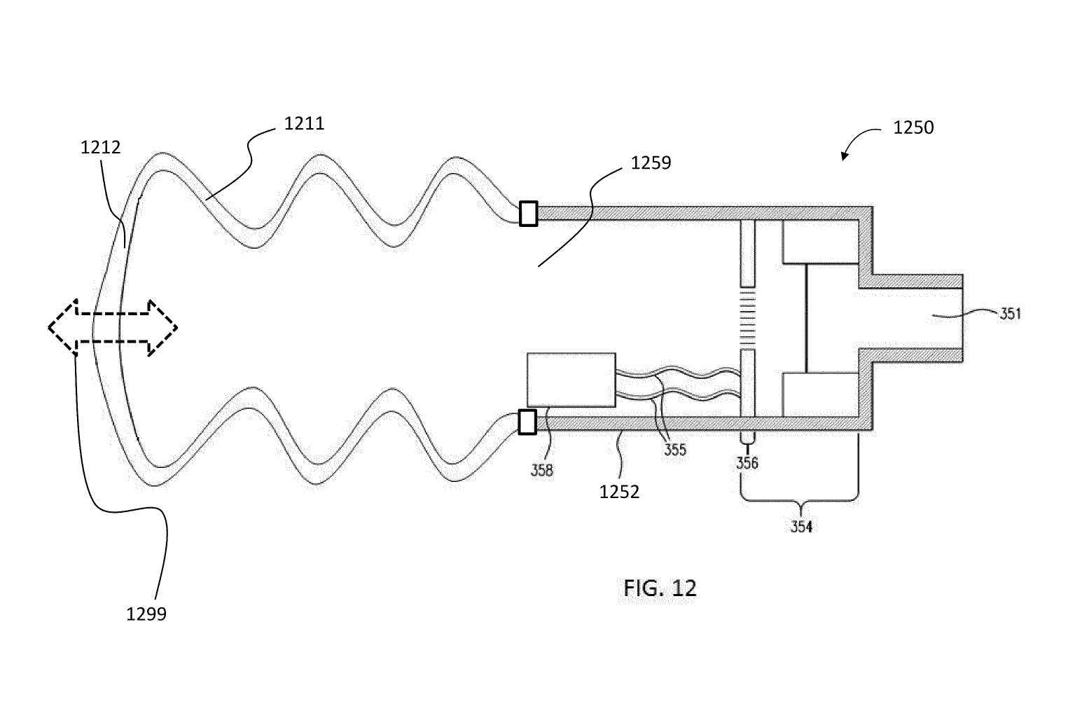

FIG. 12 depicts a cross-sectional view of a portion of an exemplary sensor assembly 1250 that corresponds to sensor assembly 350 of FIG. 4. As can be seen, the sensor assembly 1250 includes housing 1252 having one port 351 that opens to receptor 330 as detailed above.

FIG. 12 further depicts a schematic of adaptive volume structure 1211 that is also a part of sensor assembly 1250. It is noted that the embodiment of the adaptive volume structure 1211 in FIG. 12 is merely exemplary and presented in quasi-functional terms. As will be detailed below, additional structure can be utilized in the adaptive volume structure 1211 to enhance or otherwise provide utilitarian value with respect to long-term implantation in a recipient.

Like reference numbers of FIG. 12 correspond to like reference numbers of FIG. 4 (housing 1252 corresponding to housing 352 save for the addition of the adaptive volume structure 1211). Accordingly, elements 1211 and 1252 make up the components of the black box 410 of FIG. 4 and have the functionality thereof. Also, with reference to FIG. 6, reference 652 corresponds to the housing 1252 in combination with adaptive volume structure 1211 depicted in FIG. 12. Volume 659 of FIG. 6 corresponds to volume 1259.

Adaptive volume structure 1211 is constructed utilizing a material that moves in a manner analogous to an accordion. By way of example only and not by way of limitation, the walls of the adaptive volume structure 1211 are constructed of flexibly corrugated sheet(s) that enable the back wall 1212 to move in the direction of arrow 1299, thereby varying the size of the volume 1259. Accordingly, dashed arrow 1299 corresponds to dashed arrow 699, and likewise represents the expandability and contractibility of the structure 1211 and thus the volume 1259 (the back volume). As with the diaphragms of the embodiments of FIGS. 7A to 10, the adaptive volume structure 1211 is configured to expand and contract such that the volume of the back volume of the microphone 354 is expanded and contracted independent of movement of the membrane 357.

Alternatively and/or in addition to this, the adaptive volume structure 1211 can be configured of material that expands and/or contracts in a radial direction relative to the longitudinal axis of the housing 1252 with a change in ambient pressure outside the adaptive volume 1259. By way of example only and not by way of limitation, the walls 1211 can be extensions of the walls of housing 1252, where the walls collapse inward and/or expand outward toward/away from the longitudinal axis with pressure changes to equalize the pressure inside the adaptive volume 1259 with the pressure outside the adaptive volume 1259 (which can be the pressure of the ambient environment in embodiments where the adaptive volume 1259 encompasses both the front and back volumes (the combined front and back volumes)).

In an exemplary embodiment, the adaptive volume structure 1211 can be a balloon-type structure having a material that stretches and contracts with changing pressure. In this regard, in an exemplary embodiment, the adaptive volume structure 1211 can have a functionality analogous to a balloon that is “blown up” at sea level to perhaps one-quarter capacity, and then taken to a higher elevation, where the balloon expands, thereby increasing the size of the internal volume of the balloon, but equalizing the pressure inside the balloon with the ambient pressure.

In an exemplary embodiment, structural components can be utilized to limit the expansion and/or contraction of an adaptive volume structure 1211. By way of example through analogy only and not by way of limitation, in an exemplary embodiment, such a structure can limit the expansion of the balloon-like embodiment so that regardless of the pressure decrease, the balloon will only expand to a given volume, thereby preventing the balloon from bursting or the like or otherwise taking up too much room within the middle ear of the recipient.

In an exemplary embodiment, the adaptive volume structure is configured to both expand and/or contract in the axial direction and the radial direction of the longitudinal axis of the housing 1259 to vary the volume 1259 of the sensor 1250.

With continued reference to the embodiment of FIG. 12, that embodiment presents a compliant back cavity enclosure for the microphone 354 which can adapt the volume 1259 thereof to achieve the pressure equalizations detailed herein/maintain the membrane 357 at the neutral position. In an exemplary embodiment, the combined structure 1211 and 1252 is located entirely in the middle ear (corresponding to the location of sensor 350 of FIG. 3B). Accordingly, in an exemplary embodiment, the adaptive volume 1259 is entirely located in the middle ear of the recipient. In an exemplary embodiment, the combined structure 1211 and 1252 establishes a hermetically enclosed volume 1259 where the size of the volume is variable.

In an exemplary embodiment, the structure of 1211 is titanium (including a titanium alloy). Any material that can be sufficiently flexible but also have a sufficient duty cycle to provide long-term implantation of a prosthesis including the sensor 1250 of FIG. 12 can be utilized providing that the teachings detailed herein and/or variations thereof can be practiced. In an exemplary embodiment, the material is also biocompatible and can enable a hermetic seal to be established between the diaphragm and component to which it is attached.

In an exemplary embodiment, the structure 1211 is substantially rotationally symmetric about the longitudinal axis thereof (and as is the case with some embodiments of the adaptive volume structures 711, 811, 911 and 1011 and assembly 1020 detailed above) and/or the longitudinal axis of the housing 1252 (as can be the case with housing 1252.) Accordingly, in an exemplary embodiment, the structure 1211 has a circular cross-section lying on a plane normal to the longitudinal axis (as is the case with housing 1252). That said, in an alternate embodiment, the structure 1211 can have a rectangular (e.g., square) cross-section (as is the case with some embodiments of the adaptive volume structures 711, 811, 911 and 1011 and assembly 1020 detailed above). Any configuration of the structure 1211 that can enable the teachings detailed herein and are variations thereof to be practiced can be utilized in at least some embodiments.

Further, it is noted that while the embodiment of FIG. 12 depicts a configuration where the adaptive volume structure 1211 extends in the direction of the longitudinal axis of the housing 1252, in an alternate embodiment, the adaptive volume structure 1211 can extend at an angle (oblique or right angle, etc.) from that longitudinal axis. By way of example only and not by way of limitation in an exemplary embodiment, the housing 1252 can include a dogleg that changes the direction of extension of the housing 90°, from which the structure 1211 extends. Thus, the structure 1211 would be oriented 90° from that depicted in FIG. 12.

In an exemplary embodiment, the back volume of the sensor 1250 (the volume “to the left” of membrane 357-1211) can be smaller, about the same size, or larger (including substantially larger) than that of the front volume (volume “to the right” of membrane 357 the internal volume of the receptor 330, the internal volume of tube 340 and the portion of the sensor 1250 inside housing 1252 not including portion 359 (with reference to FIG. 3)), when the static pressures in the two volumes are equalized at an initial pressurization (e.g., 0.8 bars). In an exemplary embodiment, the size of the back volume is about ½, ⅔rds, the same as, two times, three times, four times, five times or more the size of the front volume when the static pressures are equalized at an initial pressurization (e.g., 0.8 bars). Any ratio of volumes of the back volume, which is a variable volume, to the front volume, which is a constant volume (or at least an effectively constant volume in that the movement of the diaphragm is negligible relative to changing the volume of the front volume) that can enable the teachings detailed herein and/or variations thereof to be practiced can be utilized in at least some embodiments.

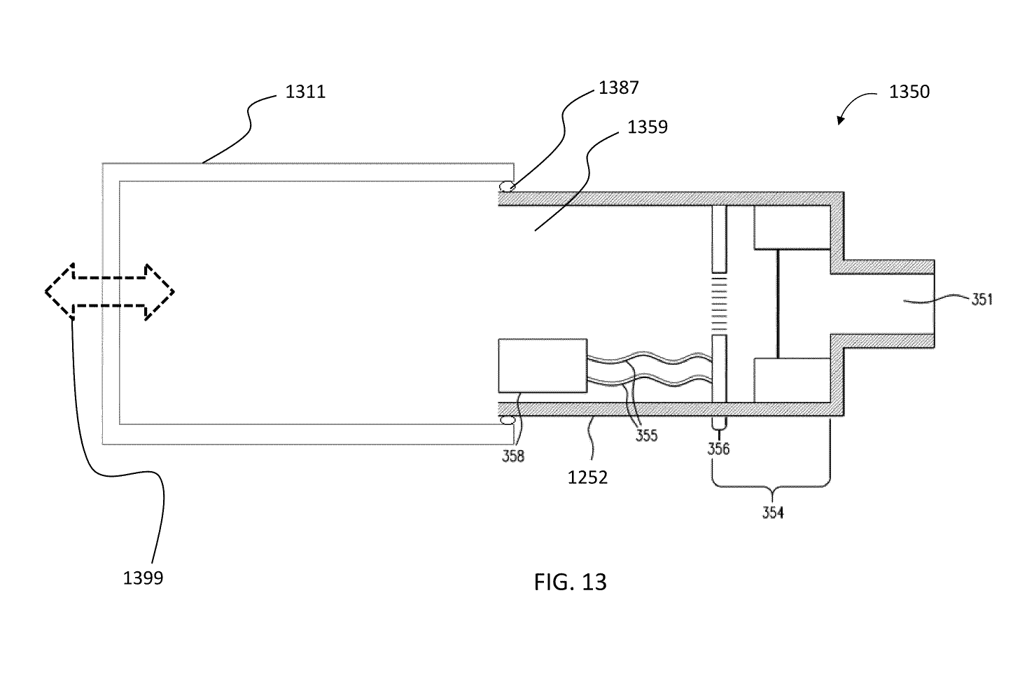

FIG. 13 depicts an alternate embodiment of the functional arrangement represented by FIG. 6. Here, instead of an accordion structure, the adaptive volume structure 1311 is a substantially rigid structure configured to move in a reciprocating manner represented by arrow 1399 along the longitudinal axis of housing 1252, thereby varying the volume 1359 of the sensor 1350. More specifically, as can be seen, the seal 1387 is located in between the outer walls of the housing 1252 and the inner walls of the adaptive volume structure 1311. When the ambient pressure decreases, the adaptive volume structure 1311 extends away from the housing 1252, thereby increasing the size of the volume 1359, and thus decreasing the pressure therein, thereby equalizing the pressure of the back volume with the front volume and thus returning the membrane 357 to the neutral position and/or equalizing the pressure of the combined front and back volumes with the pressure of the ambient environment and thus returning the diaphragm(s) 334 to the neutral position.

In an alternate embodiment, the adaptive volume structure 1311 can be configured as a piston to move to the left and to the right inside the housing 1252. Again, as with the embodiment of FIG. 12, structure can be utilized in the embodiment of FIG. 13 to limit movement of the adaptive volume structure 1311.

It is noted that like functionalities of the embodiment of FIG. 13 correspond to like functionalities detailed above with respect to the embodiment of FIG. 12 and the other embodiments, just as is the case with the embodiment of FIG. 12. In this regard, in an exemplary embodiment, the combined structure 1311 and 1252 is configured to be located entirely in the middle ear of the recipient, concomitant with the pertinent components of the schematic of FIG. 3B.

As can be seen from the embodiments of FIGS. 12 and 13, in an exemplary embodiment, the adaptive volume structure is part of a single unit that includes the microphone 354. As can be seen from the embodiments of FIGS. 12 and 13, in an exemplary embodiment, sensor 1250 and sensor 1350 are part of a single unit, where the adaptive volume structure is part of that single unit. This is as contrasted to the embodiments of FIGS. 7A-11 detailed above, where the adaptive volume structure is part of the unit that is separate from a unit that contains the microphone 354.

As noted above, in at least some embodiments, tube 501 extends from a location proximate the cochlea to a location behind and/or above the ear canal of the recipient between the mastoid bone and the outer skin of the recipient. Owing to the fact that the tube 501 must at least somewhat conform to the relevant topography of the recipient (e.g., must curve about the skull, etc.), the tube is configured to be sufficiently flexible to enable application in the recipient in accordance therewith. In an exemplary embodiment, the tube 501 extends a distance of 90 mm or thereabouts. An exemplary embodiment of the tube 501 having utilitarian value with respect to the other embodiments detailed herein and are variations thereof will now be detailed.

In an exemplary embodiment, tube 501 is a micro tube made entirely of a titanium alloy, and is embedded in a silicone shell. That said, in an alternative embodiment, the tube can be made out of other metallic materials, such as gold. In an exemplary embodiment, the tube has sufficiently high mechanical compliance to be compatible with insertion of the stimulating assembly into a cochlea during a surgical operation, as the tube 501 extends from the stimulating assembly to the receiver-stimulator of the cochlear implant in at least some embodiments. In an exemplary embodiment, the micro tube has an outer diameter of about 0.5 mm, and an interior diameter of about 0.3 mm. Any geometry that can enable the teachings detailed herein and/or variations thereof can utilize in at least some embodiments.

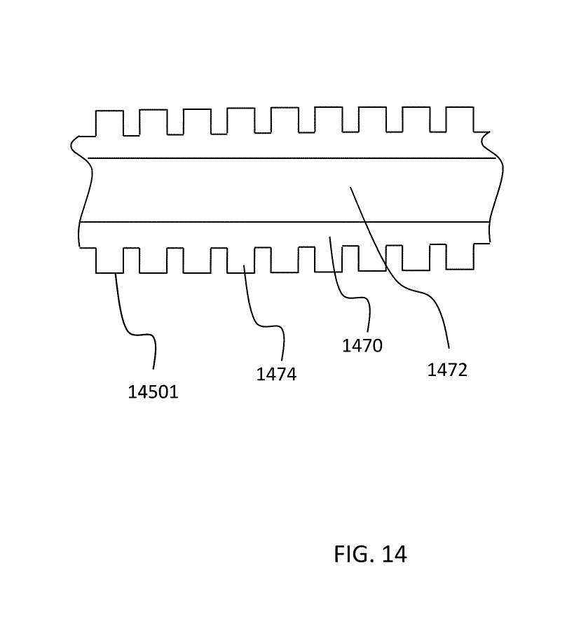

FIG. 14 depicts an exemplary embodiment of a cross-section of a portion of an exemplary micro tube 14501 corresponding to the micro tube 501 detailed above. As can be seen, micro tube 14501 includes a tube wall 1470 that establishes an internal conduit 1472 via the inside of the tube wall 1470 (which can have an internal diameter of about 0.1 mm, 0.2 mm, 0.3 mm, 0.4 mm, 0.5 mm or any value or range of values therebetween in about 0.01 mm increments).

Also as can be seen in FIG. 14, the micro tube 14501 includes corrugations 1474. In an exemplary embodiment, the corrugations are configured so as to limit the maximum bending radius of the micro tube 14501 and/or reduce the bending stiffness of the tube That is, depending on various features of the micro tube (material selection, wall thickness, conduit diameter thickness, etc.), there will be a radius at which if the micro tube is bent to a radius lower than the given radius, rupture or collapse of the conduit 1472 might result. The corrugations 1474 aid in preventing this from occurring.

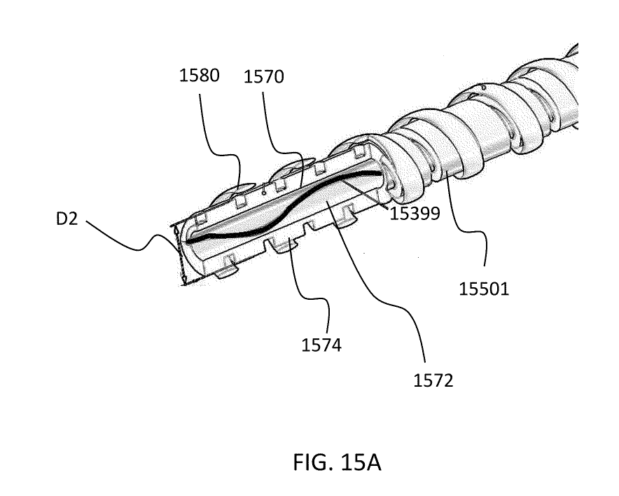

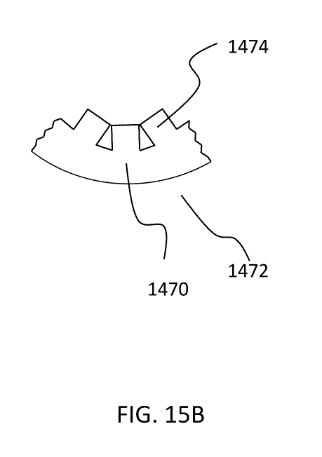

FIG. 15A depicts an isometric view of an exemplary embodiment of a micro tube 15501 based on the functional diagram of FIG. 14, where element 15501 corresponds to element 14501 of FIG. 14. FIG. 15A depicts a cut-out portion (lower left) of the micro tube depicting additional features of an exemplary micro tube. As can be seen, micro tube 15501 includes a tube wall 1570 (corresponding to wall 1470 above) that establishes an internal conduit 1572 (corresponding to conduit 1472 above) via the inside of the tube wall 1570 (corresponding to the tube wall 1470 detailed above). Also as can be seen in FIG. 15A, the micro tube 15501 includes corrugations 1574. In an exemplary embodiment, the corrugations are configured to function according to the corrugations 1474 detailed above.

FIG. 15A depicts diameter D2, which can be about 0.2 mm, 0.3 mm, 0.4 mm, 0.5 mm, 0.6 mm, 0.7 mm, 0.8 mm or any value or range of values therebetween in about 0.01 mm increments).

FIG. 15A depicts electrical lead 15399, which corresponds to electrical lead(s) 399 detailed above, which transfers the transduced energy from the microphone 354 ultimately to the receiver stimulator 180 of the cochlear implant 100 (or to another pertinent component in an alternate embodiment of a different type of hearing prosthesis). As can be seen, in the exemplary embodiment of FIG. 15A, the electrical lead 15399 extends through the conduit 1572. Accordingly, in an exemplary embodiment, the micro tube 15501 provides a conduit and a protective “armored” path for the lead 15399 to extend from the microphone 354 to the receiver stimulator.

Still with reference to FIG. 15 A, it can be seen that electrical lead(s) 1580 spirals about the outside of the microtube 15501. In an exemplary embodiment, electrical lead(s) 1580 are leads that extend from the electrodes (or other stimulating device) of the stimulator array to the receiver stimulator. In this regard, it is noted that in at least some embodiments, these electrical leads 1580 can create electromagnetic interference with respect to the lead 399 running from microphone 354 to the receiver stimulator (even if placed in a non-spiral configuration). Accordingly, in an exemplary embodiment, there is additional utilitarian value in running leads 15399 though the conduit 1572, because running the leads 15399 through the conduit provides enhanced electromagnetic interference (EMI) shielding for the leads. For example, the material of the micro tube and/or the configuration of the structure of the micro tube is such that the electrical leads 15399 are subjected to less EMI relative to that which would be the case if the lead 15399 ran outside the micro tube (parallel to and/or concentrically with the leads 1580).

That said, in at least some embodiments, the spiraling of the leads 1580 can provide utilitarian value with respect to reducing EMI induced into lead 15399 relative to that which be the case if the leads 1580 were run parallel to the micro tube 15501.

It is noted that as with other elements of the components detailed herein, both the micro tube 15501 and the leads 1580 can be embedded in elastic (e.g., highly elastic) silicone adhesive and/or other biocompatible materials.

It is noted that in alternate embodiments, other transmission devices can utilize to communicate between the microphone 354 and the receiver stimulator. By way of example only and not by way of limitation, fiber optics can be utilized. Still, in such instances, utilizing the conduit 1572 can have utilitarian value with respect to the armored features afforded thereby.

Is further noted that routing of the leads 15399 through the conduit 1572 can have utilitarian value with respect to “feeding through” the leads 15399 into the receiver stimulator. Because the interface between the receiver stimulator and the micro tube is established by these two components, the leads 15399 simply pass through into the receiver stimulator from the micro tube without the need for an individual feed through. This is also the case with respect to “feeding through” the leads 153999 into the housing 752. Because the interface between the housing 752 in the micro tube is established by these two components (a hermetic seal is already established by these two components), the leads 15399 simply pass through into the housing from the micro tube, again without the need for an individual feed through. This can have utilitarian value with respect to the fact that the housing 752 is relatively smaller than the receiver stimulator.

FIG. 15B depicts an exemplary phenomenon where the corrugations 1474 prevent further bending to a radius lower than that depicted in the figure. FIG. 15B depicts a portion of the cross-sectional view of FIG. 14, specifically, the upper cross-section of the tube wall 1470, with the conduit 1472 being indicated as open space in FIG. 15B. As can be seen, the micro tube has been bent in a radius such that the outer ends of the corrugations contact adjacent corrugations, thus preventing, or at least frustrating, the micro-tube from being bent to a smaller radius (which could induce failure, as noted above). More accurately, the configuration of FIGS. 14 and 15A and 15B can be characterized by a micro tube that is relatively easily flexed to radiuses above a certain value, and relatively more difficultly flexed to radius below a certain value (because the tube can be flexed below the pertinent radius, if only resulting in failure). Along these lines, in an exemplary embodiment, the micro tube 14501 can be considered as being a tube that provides a built in warning feature to a surgeon or the like implanting a prosthesis utilizing that micro tube to not bend the micro tube any further, where the warning is a rapid increase in resistance to bending owing to the corrugations contacting one another is depicted by way of example only and not by way limitation in FIG. 15B.

In an exemplary embodiment, the heights and/or the widths and/or the spacing between the individual corrugations is set to control the radius that is the demarcation between that which the micro tube can be more easily and less easily flexed. By way of example only and not by way of limitation, all other facets being equal, corrugations that are located further from one another will result in a higher limit bending radius than corrugations that are located closer to one another, corrugations having a high height will result in a lower limit bending radius relative to tubes that have corrugations having a lower height, corrugations having a longer length will result in a lower limit bending radius relative to telling corrugations having a lower length.

Some exemplary methods according to some exemplary embodiments will now be described.

An exemplary embodiment includes an exemplary method of adapting internal pressure of a first volume of an implanted medical device to a pressure of an ambient environment (e.g., the pressure inside the cochlea) by automatically adjusting a size of a second volume separate from the first volume. In an exemplary embodiment, this method is executed utilizing the sensor 750 detailed above, where the first volume is the volume inside housing 752, and the second volume is the volume (the hermetic volume) of adaptive volume structure 710, 810, 910 or 1010 detailed above. By “automatically,” it is meant that the size of the second volume is adjusted without human intervention.

With respect to the aforementioned exemplary method when implemented in the cochlear implant according to FIG. 11, the first volume is a volume that is proximate a cochlea of the recipient (the volume of the housing 752 “to the left” of the membrane 357) when the housing is located in the middle ear of the recipient according to FIG. 3B). The second volume (the hermetic volume of the adaptive volume structure located in the receiver-stimulator of the cochlear implant), is a volume that extends to a location between an outer skin of the recipient and an outer surface of a mastoid bone of a recipient.

In another exemplary embodiment, there is an exemplary method executed utilizing any of sensors 750, 1250 and/or 1350, that entails automatically (i.e., without human intervention) maintaining a neutral position of a membrane (e.g., membrane 357) of an implanted microphone (e.g., microphone 354). The method is executed in a device where the membrane separates a front volume from a back volume of the implanted microphone, where the front volume and back volume are fluidically isolated from one another. The method is executed when a pressure of the ambient environment in which the microphone is located changes. The method is executed by automatically adjusting the size in the back volume to at least substantially equalize the pressure in the back volume with the pressure in the front volume (which has changed due to the change in pressure of the ambient environment) and/or to at least substantially equalize the pressure in the combined front and back volume with the pressure of the ambient environment.

In an exemplary embodiment, the device in which the aforementioned method is executed is such that the front volume and the back volume are hermetically isolated volumes relative to the ambient environment of the implanted microphone. Consistent with sensors 750, 1250 and 1350 that have a receptor 330 located in the cochlea, the front volume is a volume that extends at least partially into a cochlea of the recipient, and the back volume is a volume that extends at least partially in an extra-cochlear environment of the recipient.

In an exemplary embodiment executed in a cochlear implant according to FIG. 11, the aforementioned method is executed in a device where the back volume extends to a location between an outer skin of the recipient and an outer surface of a mastoid bone of the recipient. Further in this regard, one or more or all of the aforementioned methods can be executed in conjunction with a method that entails receiving an electromagnetic signal at a first location transcutaneously transmitted from outside a recipient to an implanted medical device that include the microphone. In an exemplary embodiment, the signal can be a signal that includes energy transmitted from the external component of the cochlear implant to the internal component of the cochlear implant to recharge the battery and/or charging capacitor of the cochlear implant. In an exemplary embodiment of this method, the signal can be a signal containing information that controls or otherwise causes the cochlear implant to evoke a hearing percept in a given manner.

In an exemplary embodiment, the first location is a location of the primary internal coil of the cochlear implant. The method further includes at least one of expanding or contracting the back volume at a location at least one of at or proximate the first location. In an exemplary embodiment, this can be accomplished utilizing adaptive volume structures that are located in the receiver-stimulator of the cochlear implant proximate to the primary internal coil, as detailed above with respect to the embodiment of FIG. 11. The method is executed under a regime where the front volume is remote from at least a portion of the back volume, as is the case with the embodiment of FIG. 11.

Some exemplary performance features of the adaptive volume structures detailed herein and/or variations thereof will now be described.

In at least some embodiments, the adaptive volume structures detailed herein are configured to maintain the membrane 357 at a location where the sensitivity of the microphone 354 is relatively constant. By way of example only and not by way of limitation, such locations are deflections of the membrane 357 that are smaller than the membrane thickness (e.g., about 90%, 80%, 70%, 60%, 50%, 40%, 30%, 20% and/or 10% of the membrane thickness or any value or range of values therebetween in about 1% increments). More specifically, when the membrane is deflected away from the neutral position a significant amount, the response of the microphone 354 becomes non-linear and a relatively significant decrease in the sensing performance of the microphone 354 can occur. Accordingly, exemplary embodiments utilizing the adaptive volume structures detailed herein and variations thereof are configured to limit deflection of the membrane 357 and/or diaphragm(s) 334 due to changes in ambient pressure to deflections where the microphone response still remains substantially linear (including linear), and the sensing performance of the microphone 354 due to pressure changes is effectively maintained/not degraded.

At least some embodiments according the teachings detailed herein and are variations thereof are configured to achieve the above noted performance characteristics for changes in ambient pressure ranges ranging from 0.7 bars to 1.2 bars. Accordingly, by way of example only and not by way of limitation, in at least some embodiments, an acoustic sensitivity of an inner ear sensor such as the sensor 750, 1250 or 1350 detailed above and or variations thereof will remain effectively constant/substantially constant (including constant) within a pressure range of about 0.6 bars to about 1.3 bars, about 0.7 bars to about 1.2 bars, about 0.8 bars to about 1.1 bars, about 0.9 bars to about 1.0 bars, or within a range from about 0.6 bars to about 1.2 bars or any range therein in about 0.01 bar increments.

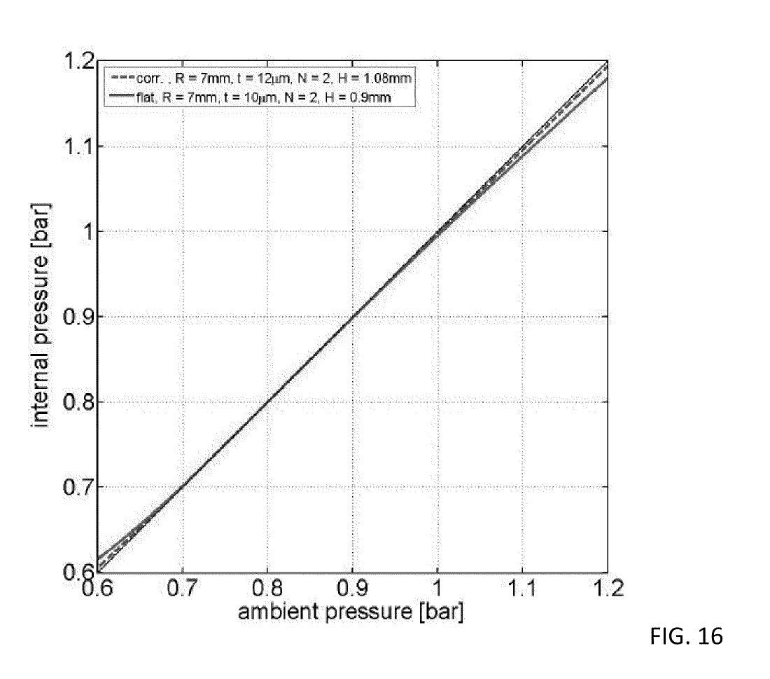

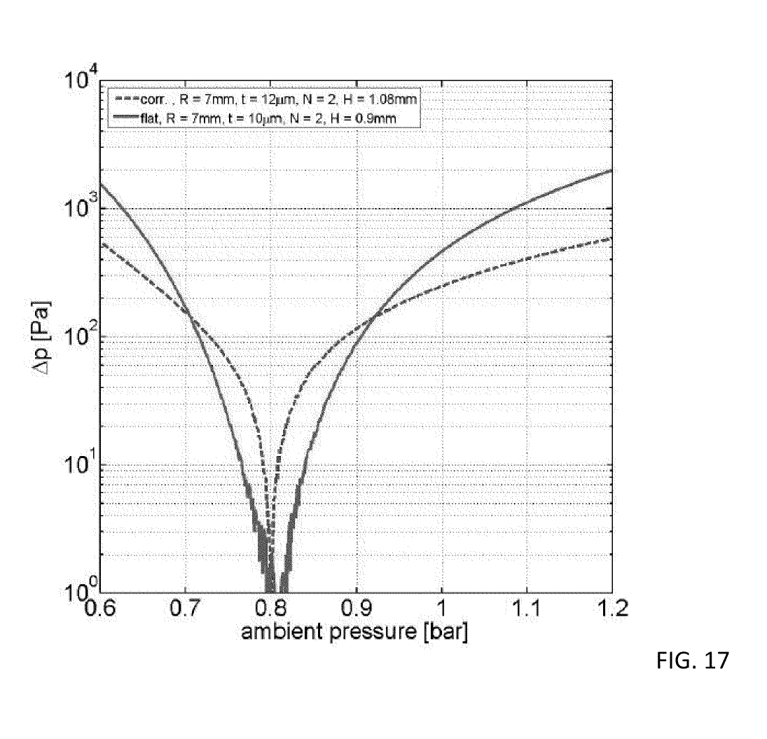

FIG. 16 presents an exemplary graph according to some exemplary performance characteristics of some exemplary systems implementing the teachings detailed herein and/or variations thereof. Specifically, FIG. 16 presents a graph of performance characteristics for two separate exemplary embodiments of the adaptive volume structure 810 of FIG. 7 detailed above having four (4) diaphragms. The first exemplary embodiment is represented by the dashed line, and utilizes corrugated diaphragms having an unclamped radius of 7 mm and a thickness of 12 μm. The height of the diaphragm stack is 1.08 mm. The number “N” in FIG. 16 indicates two (2) diaphragm pairs (i.e., the embodiment of FIG. 8). The second exemplary embodiment is represented by the solid line, and utilizes flat diaphragms also having an unclamped radius of 7 mm, but a thickness of 10 micrometers. The overall height of the diaphragm stack is 0.9 mm. Also depicted in the graph in FIG. 16 is a line indicating perfect pressure equalization (the line extending exactly from the 0.6/0.6 coordinate to exactly the 1.2/1.2 coordinate). The graph in FIG. 16 plots internal pressure of the back volume of any of the sensors detailed herein and/or variations thereof versus ambient pressure change. The performance characteristics indicated in FIG. 16 is for a sensor where the back volume and front volumes were set at an initial internal pressure of 0.8 bars. It is further noted that all performance characteristics detailed herein and are variations thereof are for sensors having a back volume in front volume set at an initial internal pressure of 0.8 bars unless otherwise noted.

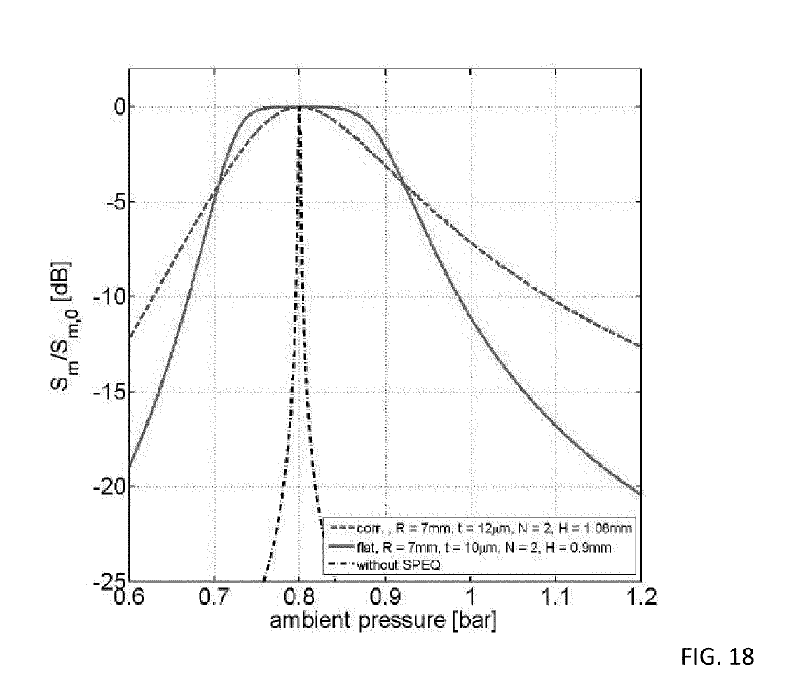

It is noted that different configurations of diaphragms can have different utilitarian value depending on a given scenario. By way of example only and not by way of limitation, a corrugated diaphragm having a thickness of about 12 μm can provide better pressure equalization performance at higher ambient pressure deviations from the initial internal pressure (e.g., 0.8 bars) than a flat diaphragm having a thickness of about 10 μm, all other things being equal. Conversely, a flat diaphragm having a thickness of about 10 μm can provide better pressure equalization at small deviations. Such phenomenon can be seen from FIG. 17, which presents performance data for a sensor having an adaptive volume structure 810, which depicts the remaining pressure difference across the membrane after equalization of the various deviations from the initial internal pressure.

As is noted in the graphs, embodiments can utilize flat diaphragms or corrugated diaphragms. In an exemplary embodiment, there is an adaptive volume structure according to any as detailed herein and/or variations thereof that utilizes a combination of flat and corrugated diaphragms. By way of example only and not by way limitation, with reference to the stack of FIG. 8, a first adaptive volume structure 710 can utilize corrugated diaphragms, and a second adaptive volume structure 710 located on the top or bottom can utilize flat diaphragms. Alternatively and/or in addition to this, a given adaptive volume structure 710 can use one corrugated diaphragm and one flat diaphragm. In at least some exemplary embodiments according to these alternate embodiments, the utilitarian value achieved by utilization of the corrugated diaphragms can be combined with utilitarian value achieved by utilizing the flat diaphragms.

The behavior of the various embodiments variously utilizing corrugated diaphragms and flat diaphragms reflects the stiffness characteristics of a corrugated diaphragm with an increasing diaphragm deflection. This can be because the corrugated diaphragm is stiffer than the flat diaphragm for small deflections. However, because of the larger linear operating ranges the corrugated diaphragm is more compliant at higher deflections. Accordingly, in an exemplary embodiment in which the sensors are expected to be utilized over a wide range of ambient pressures (e.g. 0.6 bars to 1.2 bars), the adaptive volume structures utilized in the sensors detailed herein and are variations thereof utilize corrugated diaphragms having thickness of 12 micrometers resulting in a pressure load it is reduced by approximately a factor of four relative to that which would be the case utilizing flat diaphragms having a thickness of 10 micrometers, all other things being equal.

FIG. 18 presents an exemplary graph presenting sensor performance characteristics utilizing the various adaptive volume structures according to the teachings detailed herein and are variations thereof. As with FIGS. 16 and 17, FIG. 18 presents performance data to the embodiment of FIG. 8. FIG. 18 presents sensitivity data for changes in ambient pressure relative to the initial setting of 0.8 bars. Specifically, the ratio Sm/Sm,0 corresponds to a ratio of the sensitivity of the sensor at a given ambient pressure relative to the sensitivity of that sensor at an ambient pressure of 0.8 bars (the membrane 357 being at the neutral position). FIG. 18 also presents control data for a sensor that is not equipped with a static pressure equalization system (SPEQ System). It is noted that the data for FIG. 18 is based on the utilization of a microphone in the sensor having a membrane having a diameter of 0.5 mm and a thickness of 1 μm that is made out of single-crystal silicon.

As can be seen from the graph of FIG. 18, and adaptive volume structure utilizing flat diaphragms can result in the sensitivity of the sensor being essentially constant for pressure variations smaller than about plus or minus 5 kPa. However, over the full range of pressure variations, the embodiment utilizing the corrugated diaphragms results in a corresponding drop in sensitivity of 8 dB less than that which occurs with the flat diaphragms.

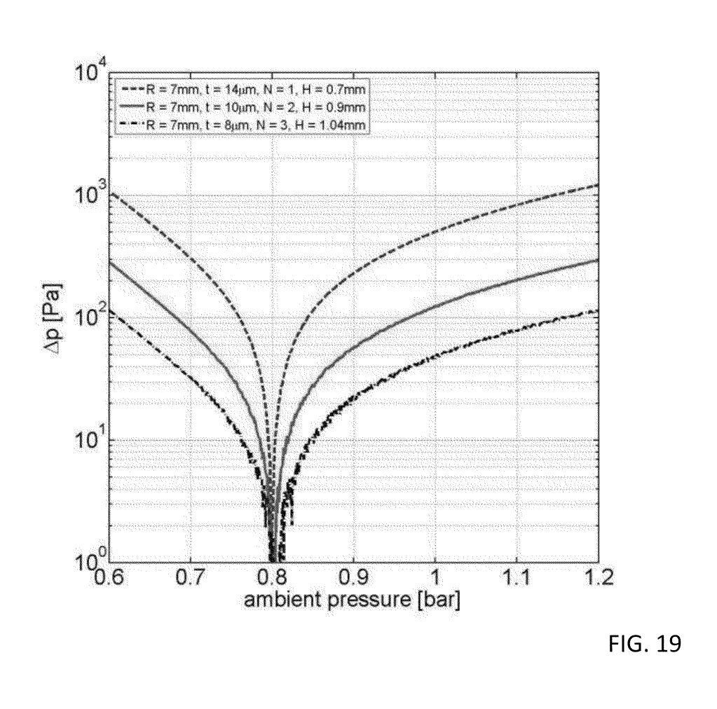

FIG. 19 presents performance characteristics for three different sensors utilizing respective different embodiments of an adaptive volume structure. More particularly, FIG. 19 presents performance data for a sensor utilizing an adaptive volume structure according to FIG. 7, represented by the dashed curve, having only a single pair of clamp diaphragms, where the thicknesses of those diaphragms are 14 μm. FIG. 19 also presents data for a sensor utilizing an adaptive volume structure according to the embodiments of FIGS. 8-10, having two pairs of clamp diaphragms, where the thicknesses of those diaphragms are 10 μm. This is represented by the solid curve. Additionally, FIG. 19 presents data for a sensor utilizing adaptive volume structure where there are three clamps diaphragm pairs, where those diaphragms thicknesses of 8 lam. This data is represented by the dotted-dashed curve. While no specific embodiments detailed herein is presented in explicit terms as having three clamps pairs, and embodiment of such can be practiced by adding a ring 821 to the adaptive volume structure 810 of FIG. 8, and an additional adaptive volume structure 710 to that ring 821. Of course, additional components such as those presented in FIGS. 9 and 10 can be added.

FIG. 19 also presents height data for the respective adaptive volume structures represented by the respective curves (indicated by the values “H” on the graphs).

In this regard, it is noted that exemplary static pressure equalization systems can include any number of combinations of adaptive volume structures. These can be arranged in a stack as presented in the embodiments of FIGS. 8, 9 and 10, and/or can be arranged in a non-stacked manner (e.g., one beside the other, one spaced away from the other, etc.), where the variable volumes thereof are manifolded together. Any arrangement of dividing structures that can enable the teachings detailed herein and or variations thereof to be practiced can utilize in at least some embodiments.

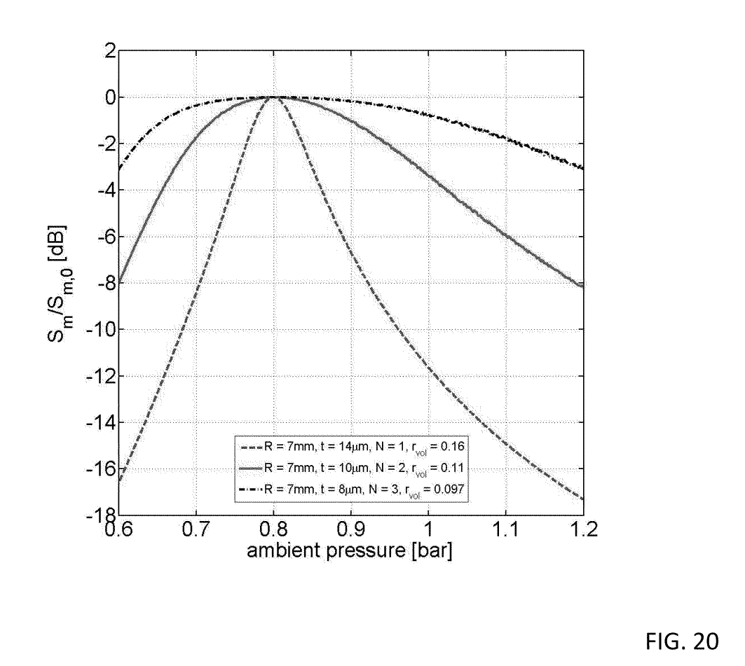

FIG. 20 presents sensor sensitivity performance data for the embodiments represented by the curves of FIG. 19, the performance data presented in FIG. 19, where Sm/Sm,0 corresponds to the ratio as detailed above. As can be seen from FIG. 20, a system utilizing three pairs of volume adapting diaphragms with respective thicknesses of the micrometers can provide sensing performance which does not change by more than about 3 dB within the ambient pressure range of six bars to 1.2 bars, again this data is for a microphone having a sound receiving membrane made out of a single crystal silicone having a diameter of 0.5 mm and the thickness of one micron.

FIG. 20 also presents ratios of the front volume to the total volume (front volume plus back volume (the hermetic back volume)) for the exemplary embodiments represented by the various curves (rvol in FIG. 20). In this regard it is noted that embodiments detailed herein and/or variations thereof can have ratios of the front volume to the total volume (front volume plus back volume) from about 0.01 to about 0.4 or any value or range of values therebetween in 0.01 increments (e.g., about 0.1, about 0.05 to about 0.2, etc.).

It is noted that the embodiments represented by FIGS. 19 and 20 present performance data for a sensor that is configured to be fully integrated into a cochlear implant (e.g., an adaptive volume structure configured to be utilized with the embodiment of FIG. 11). In an exemplary embodiment, there is utilitarian value in establishing a system where the ratio of the front volume to the total volume is relatively small, which can be achieved by making the back volume as large as possible, or at least as large as feasible.

As noted above, some and/or all of the teachings detailed herein can be used with a hearing prosthesis, such as a cochlear implant. That said, while the embodiments detailed herein have been directed towards cochlear implants, other embodiments can be directed towards application in other types of hearing prostheses, such as by way of example, bone conduction devices (e.g., active and/or passive bone conduction devices, percutaneous bone conduction devices, etc.), direct acoustic cochlear implants, etc. Indeed, embodiments can be utilized with any type of hearing prosthesis that utilizes an implanted microphone, irrespective of where the implanted microphone is located.

Further, while embodiments detailed herein are directed towards sensors used for cochlear implants/used for intra-cochlear implementations, other embodiments can be utilized for other types of the implantable devices having volumes that are hermetically sealed, such as by way of example only and not by way of limitation, intracranial implementations intraocular implementations and/or any other intra-body dynamic pressure measurement sensors to which the teachings detailed herein and are variations thereof can be applicable.

It is noted that any disclosure with respect to one or more embodiments detailed herein can be practiced in combination with any other disclosure with respect to one or more other embodiments detailed herein.

It is noted that some embodiments include a method of utilizing a prosthesis including one or more or all of the teachings detailed herein and/or variations thereof. In this regard, it is noted that any disclosure of a device and/or system herein also corresponds to a disclosure of utilizing the device and/or system detailed herein, at least in a manner to exploit the functionality thereof. Further, it is noted that any disclosure of a method of manufacturing corresponds to a disclosure of a device and/or system resulting from that method of manufacturing. It is also noted that any disclosure of a device and/or system herein corresponds to a disclosure of manufacturing that device and/or system.

While various embodiments have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

What is claimed is:

1. A device, comprising:
an implantable sensor having a membrane displaceable in response to physical phenomena outside the sensor, wherein
the device is configured to equalize a static pressure difference between an ambient environment and a back volume of the sensor.

2. The device of claim 1, wherein:
the device is configured to adapt a size of the back volume of the sensor to a change in ambient pressure, thereby equalizing the static pressure difference.

3. The device of claim 1, wherein:
the device includes a compliant back cavity that makes up at least a portion of the back volume.

4. The device of claim 1, wherein:
the back volume includes a first volume and a second volume remote from and distinct from the first volume in fluid communication with the first volume; and
the first volume is proximate the membrane.

5. The device of claim 4, wherein:
the first volume is located in a first housing and the second volume is located in a second housing remote from the first housing.

6. The device of claim 4, wherein:
the first volume is in fluid communication with the second volume via a micro-tube.

7. The device of claim 1, wherein:
the back volume is established by a chamber bounded in part by the membrane, wherein the chamber is configured to vary the volume of the back volume in a manner beyond that resulting from displacement of the membrane.

8. The device of claim 7, wherein:
the chamber is proximate the membrane.

9. The device of claim 1, wherein:
the device includes a cochlear implant electrode array assembly, wherein the sensor and the cochlear implant electrode array are part of a single unit.

10. A device, comprising:
an implantable microphone having a membrane displaceable in response to a change in a phenomena of fluid in a cochlea induced by ambient sound, the membrane forming a portion of a boundary of a back volume of the microphone, wherein
the device is configured to expand and contract a size of the volume of the back volume independent of movement of the membrane.

11. The device of claim 10, wherein:
(i) the front and back volumes are fluidically isolated from one another, and the device is configured to expand the size of the volume of the back volume in response to an increase in static pressure on an opposite side of the diaphragm relative to the back volume, and the device is configured to contract the size of the volume of the back volume in response to a decrease in static pressure on the opposite side of the diaphragm relative to the back volume; or
(ii) the front and back volumes are in fluid communication with one another, and the device is configured to expand the size of the volume of the back volume in response to a decrease in static pressure in an ambient environment of the device, and the device is configured to contract the size of the volume of the back volume in response to an increase in static pressure in the ambient environment of the device.

12. The device of claim 10, wherein at least one of:
the device is configured such that the expansion and contraction of the size of the volume of the back volume equalizes the static pressure on the opposite side of the diaphragm with the static pressure in the back volume, wherein the volume on the opposite side of the diaphragm and the back volume are fluidically isolated from one another; or
the device is configured such that the expansion and contraction of the size of the volume of the back volume equalizes the static pressure of a volume on the opposite side of the diaphragm and the back volume with the ambient environment, wherein the volume on the opposite side of the diaphragm and the back volume are in fluid communication with one another.

13. The device of claim 10, wherein:
the microphone is part of a first unit; and
the device includes:
a second unit distinct from the first unit, the second unit being configured to expand and contract such that the volume of the back volume is expanded and contracted independent of movement of the diaphragm.

14. The device of claim 10, wherein:
the second unit is in fluid communication with the first unit via a corrugated micro-tube.

15. The device of claim 10, wherein:
the second unit includes a stack of clamped diaphragms, wherein the diaphragms are configured to deflect in first directions and second directions, thereby respectively expanding and contracting the back volume independent of the movement of the diaphragm.

16. The device of claim 10, wherein:
the microphone is part of a first unit that is configured to expand and contract such that the volume of the back volume is expanded and contracted independent of movement of the diaphragm.

17. The device of claim 16, wherein:
the first unit includes an accordion wall configured to expand and contact such that the volume of the back volume is expanded and contracted independent of movement of the diaphragm.

19. An apparatus, including:
a cochlear implant comprising the device of claim 11, wherein the cochlear implant further includes:
a cochlear implant electrode array assembly including the implantable microphone; and
a receiver-stimulator component, wherein
the volume of the back volume extends from the electrode array assembly to the receiver-stimulator component.

20. A device, comprising:
an implantable static pressure equalization system configured to equalize an internal pressure of an apparatus with a static pressure of an ambient environment, the apparatus being configured to sense a dynamic phenomenon in a recipient, the system including:
at least one diaphragm bounding a volume, wherein the diaphragm is configured to deflect in response to a change in the static pressure, thereby adjusting the size of the volume bounded by the diaphragm, wherein the system is configured such that the volume is placed in fluid communication with the apparatus, and wherein the diaphragm is sheltered by at least two substantially rigid components located on opposite sides of the diaphragm in a direction normal to a maximum diameter of the diaphragm.

21. The device of claim 20, wherein the system includes:
at least two diaphragms arranged in a stack, wherein a space between the two diaphragms is part of the volume.

22. The device of claim 20, wherein:
the system is configured to enable ingress and egress of a body fluid between the diaphragm and at least one of the substantially rigid components; and
the system is configured such that the volume is hermetically sealed from the body fluid when the volume is placed in fluid communication with the apparatus.

23. The device of claim 20, wherein:
the system is configured with a non-hermetic volume that is hermetically isolated from the volume, wherein the non-hermetic volume extends in between at least one of the substantially rigid components and the diaphragm, and wherein the non-hermetic volume is separated from the ambient environment by a silicone housing.

24. The device of claim 20, wherein:
the system includes a first sub-volume bounded by at least a first diaphragm, and a second sub-volume bounded by at least a second diaphragm, the first sub-volume being in fluid communication with the second sub-volume and collectively forming at least part of the volume, wherein the sub-volumes are arrayed in the direction normal to the maximum diameter, and wherein a first size of the first sub-volume is independent of a second size of the second sub-volume.

25. The device of claim 20, wherein the device includes an implantable component comprising:
a stack of:
the diaphragm and at least one other diaphragm;
two caps respectively corresponding to the substantially rigid components; and
a spacer spacing apart the two diaphragms; and
a silicone housing encompassing the stack of the diaphragms, the caps and the spacer.

26. The device of claim 20, further including:
a permanent magnet in the stack; and
a receiver coil, wherein
the receiver coil and the permanent magnet are also encompassed in the silicone housing.

27. A method, comprising:
automatically maintaining a neutral position of at least one of (i) a membrane of an implanted microphone having a front volume and a back volume separated by the membrane and fluidically isolated from one another in response to a change in pressure of the front volume induced by a change in pressure of an ambient environment in which the microphone is located or (ii) a flexible diaphragm of a pressure receptor that hermetically isolates an internal volume in fluid communication with the microphone with an ambient environment by automatically adjusting the size of the back volume to at least substantially equalize the pressure of at least one of the back volume or the pressure of a combined front and back volume with the pressure of the ambient environment.

28. The method of claim 27, wherein:
the front volume and the back volume are hermetically isolated volumes relative to an ambient environment of the implanted medical device.

29. The method of claim 27, wherein:
the front volume is a volume that extends at least partially into a cochlea of the recipient; and
the back volume is a volume that extends at least partially in an extra-cochlear environment of the recipient.

30. The method of claim 29, wherein:
the back volume extends to a location between an outer skin of the recipient and an outer surface of a mastoid bone of a recipient.

31. The method of claim 27, further comprising:
receiving an electromagnetic signal at a first location transcutaneously transmitted from outside a recipient to the implanted medical device; and
at least one of expanding or contracting the back volume at a location at least one of at or proximate the first location, wherein
the front volume is remote from at least a portion of the back volume.
